(19) United States
(12) Patent Application Publication
(10) Pub. No.: US 2018/0016566 A1
(43) Pub. Date: Jan. 18, 2018

(54) DETERGENT COMPOSITION COMPRISING PROTEASE AND AMYLASE VARIANTS

(71) Applicant: NOVOZYMES A/S, Bagsvaerd (DK)

(72) Inventors: Miguel Duarte Guilherme Pereira Toscano, Greve (DK); Carsten Andersen, Vaerloese (DK); Bena-Marie Lue, Holte (DK)

(73) Assignee: NOVOZYMES A/S, Bagsvaerd (DK)

(21) Appl. No.: 15/548,786

(22) PCT Filed: Feb. 3, 2016
(86) PCT No.: PCT/EP2016/052297

§ 371 Date: Aug. 4, 2017

(30) Foreign Application Priority Data
Feb. 4, 2015 (EP) 15153789.1

(51) Int. Cl. C12N 9/54; C11D 3/386; C11D 11/00; C12N 9/28

(52) U.S. Cl. CPC C12N 9/54; C12N 9/2417; C11D 3/386; C11D 11/0017

(57) ABSTRACT

The present invention relates to detergent compositions comprising protease variants and alpha-amylases or variants thereof. Furthermore, the present invention relates to methods of using the detergent compositions.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to novel compositions comprising protease variants and amylase variants, wherein the respective variants exhibit alterations relative to the parent protease and parent amylase, respectively, in one or more properties including: wash performance, detergent stability and/or storage stability. The compositions of the invention are suitable as e.g. cleaning or detergent compositions, such as laundry detergent compositions and dish wash compositions, including automatic dish wash compositions.

### Description of the Related Art

Enzymes have been used within the detergent industry as part of washing formulations for many decades. Proteases are from a commercial perspective the most relevant enzyme in such formulations, but other enzymes including lipases, amylases, cellulases, hemicellulases or mixtures of enzymes are also often used. To improve the cost and/or the performance of enzymes there is an ongoing search for enzymes with altered properties, such as increased activity at low temperatures, increased stability, increased specific activity at a given pH, altered Ca²⁺ dependency, increased stability in the presence of other detergent ingredients (e.g. bleach, surfactants etc.) etc. For instance, one family of proteases, which is often used in detergents, are the subtilases. This family has previously been further grouped into 6 different sub-groups by Siezen R J and Leunissen JAM, 1997, Protein Science, 6, 501-523. One of these sub-groups is the Subtilisin family which includes subtilases such as BPN', subtilisin 309 (SAVINASE®, Novozymes NS), subtilisin Carlsberg (ALCALASE®,Novozymes A/S), subtilisin S41 (a subtilase from the psychrophilic Antarctic Bacillus TA41, Davail S et al. 1994, The Journal of Biological Chemistry, 269(26), 99. 17448-17453) and subtilisin S39 (a subtilase from the psychrophilic Antarctic Bacillus TA39, Narinx E et al. 1997, Protein Engineering, 10 (11), pp. 1271-1279). TY145 is a subtilase from Bacillus sp. TY145, NCIMB 40339, which was first described in WO 92/17577 (Novozymes NS) and in the later application WO2004/067737 (Novozymes NS) disclosing the three-dimensional structure and the use of protein engineering to alter functionality of a TY-145 subtilase.

Other enzymes, such as the alpha-amylases, have typically been alpha-amylases from B. licheniformis, also known as Termamyl. Similar to protease ongoing search for improvements, alpha-amylases are under development.

Detergent compositions have been described, but there is a continued need for improved detergent compositions, wherein the enzymes within the detergent compositions, such as the bleaching system or chelators. Thus, it is an objective of the present invention to provide such detergent compositions.

### SUMMARY OF THE INVENTION

The present invention relates to detergent compositions comprising (i) at least one protease variant comprising a substitution at one or more positions corresponding to positions 171, 173, 175, 179, or 180 of SEQ ID NO: 1, wherein the protease variant has a sequence identity of at least 75% but less than 100% to SEQ ID NO: 1, and wherein the protease variant has protease activity, and (ii) at least one alpha-amylase having the amino acid sequence of SEQ ID NO: 2, 3, 4, 5, 6, 7, 8, or 9, or a variant thereof having a sequence identity of at least 75% but less than 100% to SEQ ID NO: 2, 3, 4, 5, 6, 7, 8, or 9, and wherein the alpha-amylase variant has alpha-amylase activity.

The present invention relates also to a method of laundering comprising laundering a fabric with a detergent composition according to the invention, preferably at a temperature of 40° C. or less, or more preferably at a temperature of 30° C. or less, or even more preferably at a temperature of 20° C. or less.

### Overview of Sequences Listing

SEQ ID NO: 1 is the amino acid sequence of the mature TY145 protease.
SEQ ID NO: 2 is the amino acid sequence of the mature SP722 alpha-amylase
SEQ ID NO: 3 is the amino acid sequence of the mature AA560 alpha-amylase
SEQ ID NO: 4 is the amino acid sequence of the mature TS23 alpha-amylase
SEQ ID NO: 5 is the amino acid sequence of the mature cytophaga alpha-amylase
SEQ ID NO: 6 is the amino acid sequence of the mature SP707 alpha-amylase
SEQ ID NO: 7 is the amino acid sequence of the mature LASB0000 alpha-amylase
SEQ ID NO: 8 is the amino acid sequence of the mature SP7.7 alpha-amylase
SEQ ID NO: 9 is the amino acid sequence of the mature LE174
SEQ ID NO: 10 is the amino acid sequence of the mature TLL lipase.

### Definitions

The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Engng. 4 (1991) 719-737 and Siezen et al. Protein Science 6 (1997) 501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Proteases of the invention are endopeptidases (EC 3.4.21). For purposes of the present invention, protease activity is determined according to the procedure described in Example 1 below. The protease variants described herein have at least 20%, e.g., at least 40%, at least 50%, at least 60%, at least 70%, at least 80%, at least 90%, at least 95%, or at least 100% of the protease activity of the mature polypeptide with SEQ ID NO: 1.

The term “alpha-amylase” means an alpha-amylase having alpha-amylase activity, i.e. the activity of alpha-1,4-glucan-4-glucanohydrolases, E.C. 3.2.1.1, which constitute a group of enzymes, catalysing hydrolysis of starch and other linear and branched 1,4-glucosidic oligo- and polysaccharides. For purposes of alpha-amylases present in the detergent compositions of the present invention, alpha-amylase activity may be determined as described in Example 1 below. The alpha-amylases described herein have at least 20%, e.g., at least 40%, at least 50%, at least 60%, at least 70%, at least 80%, at least 90%, at least 95%, or at least 100% of the alpha-amylases activity of the mature polypeptide with SEQ ID NO: 2.

The term “lipase” means a lipase having lipase activity. The lipase defined herein may be a carboxylic ester hydrolase EC 3.1.1,-, which includes activities such as EC 3.1.1.3 triacylglycerol lipase, EC 3.1.1.4 phospholipase A2, EC 3.1.1.5 lysophospholipase, EC 3.1.1.26 galactolipase, EC 3.1.1.32 phospholipase Al, EC 3.1.1.73 feruloyl esterase.

The term “protease variant” means a protease having protease activity comprising an alteration, i.e., a substitution, insertion, and/or deletion, preferably substitution, at one or more (or one or several) positions compared to its parent which is a protease having the identical amino acid sequence of said variant but not having the alterations at one or more of said specified positions. Similarly, the term “alpha-amylase variant” means an alpha-amylase having alpha-amylase activity comprising an alteration, i.e., a substitution, insertion, and/or deletion, at one or more (e.g., several) positions as compared to a “parent alpha-amylase”. A substitution means a replacement of an amino acid occupying a position with a different amino acid; a deletion means removal of an amino acid occupying a position; and an insertion means adding amino acids e.g. 1 to 10 amino acids, preferably 1-3 amino acids adjacent to an amino acid occupying a position. Amino acid substitutions may exchange a native amino acid for another naturally-occurring amino acid, or for a non-naturally-occurring amino acid derivative. The alpha-amylase variants have at least 20%, e.g., at least 40%, at least 50%, at least 60%, at least 70%, at least 80%, at least 90%, at least 95%, or at least 100% of the alpha-amylase activity of the mature parent alpha-amylase from which they have been derived.

The term “isolated variant” means a variant that is modified by the hand of man. In one aspect, the variant is at least 1% pure, e.g., at least 5% pure, at least 10% pure, at least 20% pure, at least 40% pure, at least 60% pure, at least 80% pure, and at least 90% pure, as determined by SDS-PAGE.

The term “parent protease” means a protease to which an alteration is made to produce the protease variants. Thus, the parent protease is a protease having the identical amino acid sequence of said protease variant but not having the alterations at one or more of said specified positions. It will be understood, that in the present context the expression “having identical amino acid sequence” relates to 100% sequence identity. Similarly, the term “parent alpha-amylase” refers to an alpha-amylase to which an alteration is made to produce the alpha-amylase variant. Thus, the parent alpha-amylase is an alpha-amylase having the identical amino acid sequence of said alpha-amylase variant but not having the alterations at one or more of said specified positions.

Therefore, the parent, being either a parent protease or parent alpha-amylase, may be a naturally occurring (wild-type) polypeptide or a variant thereof. In a particular embodiment, the parent is a protease with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 1. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 2. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 3. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 4. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 5. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 6. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 7. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 8. In a particular embodiment, the parent is an alpha-amylase with at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, e. g. at least 99.1%, at least 99.2%, at least 99.3%, at least 99.4%, at least 99.5%, at least 99.6 or 100% identity to a polypeptide with SEQ ID NO: 9.

The term “wild-type protease” means a protease expressed by a naturally occurring organism, such as a bacterium, archaea, yeast, fungus, plant or animal found in nature. An example of a wild-type protease is TY-145, i.e. the mature polypeptide with SEQ ID NO: 1.

The term “wild-type alpha-amylase” means an alpha-amylase expressed by a naturally occurring microorganism, such as a bacterium, yeast, or filamentous fungus found in nature. An example of a wild-type alpha-amylase is AA560, i.e., the polypeptide of SEQ ID NO: 3.

SEQ ID NO: 1The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

The term “operably linked” means a configuration in which a control sequence is placed at an appropriate position relative to the coding sequence of a polynucleotide such that the control sequence directs the expression of the coding sequence.

The term “control sequences” means all components necessary for the expression of a polynucleotide encoding a variant of the present invention. Each control sequence may be native or foreign to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

The term “expression” includes any step involved in the production of the variant including, but not limited to, transcription, post-transcriptional modification, translation, post-translational modification, and secretion.

The term “expression vector” means a linear or circular DNA molecule that comprises a polynucleotide encoding a variant and is operably linked to additional nucleotides that provide for its expression.

The term “transcription promoter” is used for a promoter which is a region of DNA that facilitates the transcription of a particular gene. Transcription promoters are typically located near the genes they regulate, on the same strand and upstream (towards the 5′ region of the sense strand).

The term “transcription terminator” is used for a section of the genetic sequence that marks the end of gene or operon on genomic DNA for transcription.

The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

The term “improved property” when referring to a protease variant herein, means a characteristic associated with a variant that is improved compared to the parent or compared to a protease with SEQ ID NO: 1, or compared to a protease having the identical amino acid sequence of said variant but not having the alterations at one or more of said specified positions. Such improved properties include, but are not limited to, wash performance, protease activity, thermal activity profile, thermostability, pH activity profile, pH stability, substrate/cofactor specificity, improved surface properties, product specificity, increased stability, improved stability under storage conditions, and chemical stability.

The term “improved protease activity” is defined herein as an altered protease activity (as defined above) e.g. by increased protein conversion of a protease variant displaying an alteration of the activity relative (or compared) to the activity of the parent protease, or compared to a protease with SEQ ID NO: 1, or relative to a protease having the identical amino acid sequence of said protease variant but not having the alterations at one or more of said specified positions.

The term “improved property” when referring to an alpha-amylase variant herein, refers to a characteristic associated with an alpha-amylase variant that is improved compared to the parent alpha-amylase, e.g. a parent alpha-amylase having the sequence of SEQ ID NO: 2, 3, 4, 5, 6, 7, 8, or 9, or compared to an alpha-amylase having the identical amino acid sequence of said variant but not having the alteration at one or more of said specified positions. Such improved properties include, but are not limited to, wash performance, alpha-amylase activity, thermal activity profile, thermostability, pH activity profile, pH stability, substrate specificity, improved surface properties, product specificity, increased stability, improved stability under storage conditions, and chemical stability.

The term “improved alpha-amylase activity” is defined herein as an altered alpha-amylase activity (as defined above), e.g., by increased polysaccharide conversion of an alpha-amylase variant displaying an alteration of the activity relative (or compared) to the activity of the parent alpha-amylase, or compared to an alpha-amylase with SEQ ID NO: 2, 3, 4, 5, 6, 7, 8, or 9, or relative to an alpha-amylase having the identical amino acid sequence of said alpha-amylase variant but not having the alterations at one or more of said specified positions.

The term “stability” includes storage stability and stability during use, e.g. during a wash process and reflects the stability of the protease variant according to the invention as a function of time e.g. how much activity is retained when the protease variant is kept in solution in particular in a detergent solution. The stability is influenced by many factors e.g. pH, temperature, detergent composition e.g. amount of builder, surfactants etc. The term “improved stability” or “increased stability” is defined herein as a variant being either a protease variant or an alpha-amylase variant displaying an increased stability in solutions, relative to the stability of the parent protease or parent alpha-amylase, relative to a protease or an alpha-amylase having the identical amino acid sequence of said variant but not having the alterations at one or more of said specified positions or relative to SEQ ID NO: 1, 2, 3, 4, 5, 6, 7, 8, or 9, depending on which parent polypeptide the variant has been derived from. The terms “improved stability” and “increased stability” includes “improved chemical stability”, “detergent stability” or “improved detergent stability. Enzyme stability may be measured as described in the Examples.

The term “improved chemical stability” is defined herein as a variant enzyme displaying retention of enzymatic activity after a period of incubation in the presence of a chemical or chemicals, either naturally occurring or synthetic, which reduces the enzymatic activity of the parent enzyme. Improved chemical stability may also result in variants being more able to catalyze a reaction in the presence of such chemicals. In a particular aspect of the invention the improved chemical stability is an improved stability in a detergent, in particular in a liquid detergent. The term “detergent stability” or “improved detergent stability” is in particular an improved stability of the enzyme activity when an enzyme variant is mixed into a liquid detergent formulation, especially into a liquid detergent formulation according to table 1 and then stored at temperatures between 15 and 50° C., e. g. 20° C., 30° C. or 40° C. for at least one week.

The term “improved thermal activity” means a variant displaying an altered temperature-dependent activity profile at a specific temperature relative to the temperature-dependent activity profile of the parent or relative to a polypeptide of SEQ ID NO: 1, 2, 3, 4, 5, 6, 7, 8, or 9. The thermal activity value provides a measure of the variant's efficiency in enhancing catalysis of a hydrolysis reaction over a range of temperatures.

The term “improved wash performance” is defined herein as a variant displaying an improved wash performance relative to the wash performance of the parent enzyme, relative to a polypeptide of SEQ ID NO: 1, 2, 3, 4, 5, 6, 7, 8, or 9, or relative to an enzyme having the identical amino acid sequence of said variant but not having the alterations at one or more of said specified positions e.g. by increased stain removal. The term “wash performance” includes wash performance in laundry but also e.g. in dish wash. The wash performance may be quantified as described under the definition of “wash performance” herein.

In a particular embodiment, the detergent composition of the present invention has an improved wash performance when compared to a detergent composition comprising only a protease variant or an alpha-amylase. Thus, it is believed that the detergent composition comprising both a protease and an alpha-amylase variant have a beneficial effect on wash performance. In another particular embodiment, the protease and alpha-amylase variants may show synergy and thereby, provide a detergent composition having an even further improved wash performance when compared to a detergent composition comprising only one of the enzymes. The term “detergent composition”, includes unless otherwise indicated, granular or powder-form all-purpose or heavy-duty washing agents, especially cleaning detergents; liquid, gel or paste-form all-purpose washing agents, especially the so- called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. The term “detergent composition” is not intended to be limited to compositions that contain surfactants. It is intended that in addition to the variants herein described, the detergents compositions may comprise, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and/or solubilizers.

The term “fabric” encompasses any textile material. Thus, it is intended that the term encompass garments, as well as fabrics, yarns, fibers, non-woven materials, natural materials, synthetic materials, and any other textile material.

The term “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers. The term, “textile materials” is a general term for fibers, yarn intermediates, yarn, fabrics, and products made from fabrics (e.g., garments and other articles).

The term “non-fabric detergent compositions” include non-textile surface detergent compositions, including but not limited to compositions for hard surface cleaning, such as dishwashing detergent compositions, oral detergent compositions, denture detergent compositions, and personal cleansing compositions.

The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like. The term “effective amount” of a variant refers to the quantity of variant described hereinbefore that achieves a desired level of enzymatic activity, e.g., in a defined detergent composition. In one embodiment, the effective amount of a protease variant is the same effective amount of an alpha-amylase, such as an alpha-amylase variant. In another embodiment, the effective amount of a protease variant is different than the effective amount of an alpha-amylase, such as an alpha-amylase variant, e.g., the effective amount of a protease variant may be more or may be less than the effective amount of an alpha-amylase, such as an alpha-amylase variant.

The term “water hardness” or “degree of hardness” or “dH” or “°dH” as used herein refers to German degrees of hardness. One degree is defined as 10 milligrams of calcium oxide per litre of water.

The term “relevant washing conditions” is used herein to indicate the conditions, particularly washing temperature, time, washing mechanics, detergent concentration, type of detergent and water hardness, actually used in households in a detergent market segment.

The term “adjunct materials” means any liquid, solid or gaseous material selected for the particular type of detergent composition desired and the form of the product (e.g., liquid, granule, powder, bar, paste, spray, tablet, gel, or foam composition), which materials are also preferably compatible with the enzymes used in the composition. In some embodiments, granular compositions are in “compact” form, while in other embodiments, the liquid compositions are in a “concentrated” form.

The term “stain removing enzyme” as used herein, describes an enzyme that aids the removal of a stain or soil from a fabric or a hard surface. Stain removing enzymes act on specific substrates, e.g., protease on protein, amylase on starch, lipase and cutinase on lipids (fats and oils), pectinase on pectin and hemicellulases on hemicellulose. Stains are often depositions of complex mixtures of different components which either results in a local discolouration of the material by itself or which leaves a sticky surface on the object which may attract soils dissolved in the washing liquor thereby resulting in discolouration of the stained area. When an enzyme acts on its specific substrate present in a stain the enzyme degrades or partially degrades its substrate thereby aiding the removal of soils and stain components associated with the substrate during the washing process. For example, when a protease acts on a grass stain it degrades the protein components in the grass and allows the green/brown colour to be released during washing.

The term “reduced amount” means in this context that the amount of the component is smaller than the amount which would be used in a reference process under otherwise the same conditions. In a preferred embodiment the amount is reduced by, e.g., at least 5%, such as at least 10%, at least 15%, at least 20% or as otherwise herein described.

The term “low detergent concentration” system includes detergents where less than about 800 ppm of detergent components is present in the wash water. Asian, e.g., Japanese detergents are typically considered low detergent concentration systems.

The term “medium detergent concentration” system includes detergents wherein between about 800 ppm and about 2000 ppm of detergent components is present in the wash water. North American detergents are generally considered to be medium detergent concentration systems.

The term “high detergent concentration” system includes detergents wherein greater than about 2000 ppm of detergent components is present in the wash water. European detergents are generally considered to be high detergent concentration systems.

It is within the knowledge of the skilled person to know how to align amino acid sequences in order to determine which amino acid in a particular position referred to herein “corresponds to” in another amino acid sequence not listed herein. Thus, the term “position corresponding to” as used herein, is well-known within the art.

The term “liquid laundry detergent composition” as used herein refers to a detergent composition which is in a stabilized liquid form and used in a method for laundering a fabric. Thus, the detergent composition has been formulated to be in fluid form.

The term “powder laundry detergent composition” as used herein refers to a detergent composition which is in a solid form, such as a granulate, non-dusting granulate or powder, which is used in a method for laundering a fabric.

The term “liquid dishwash detergent composition” as used herein refers to a detergent composition which is in a stabilized liquid form and used in dishwash. Dishwash may be any kind of dishwash, such as manual dishwash and such as automated dishwash (ADW).

The term “powder dishwash detergent composition” as used herein refers to a detergent composition which is in a solid form, such as a granulate, powder or compact unit and used in dishwash. A powder dishwash detergent composition is typically used in automated dishwash, but the used is not limited to such ADW, and may also be intended for used in any other kind of dishwash, such as manual dishwash.

### Conventions for Designation of Variants

For purposes of the present invention, the mature polypeptides disclosed in SEQ ID NO: 1, 2, 3, 4, 5, 6, 7, 8, and 9 are used to determine the corresponding amino acid residue in another polypeptide. The amino acid sequence of another polypeptide is aligned with the mature polypeptide disclosed in SEQ ID NO: 1, 2, 3, 4, 5, 6, 7, 8, or 9 depending on whether it is a protease or an alpha-amylase, and based on the alignment, the amino acid position number corresponding to any amino acid residue in the mature polypeptide disclosed in SEQ ID NO: 1, 2, 3, 4, 5, 6, 7, 8, or 9 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another protease can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:_39-64; Katoh and Toh, 2010, Bioinformatics 26:_1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 1, 2, 3, 4, 5, 6, 7, 8, or 9 such that traditional sequence-based comparison fails to detect their relationship (Lindahl and Elofsson, 2000, J. Mol. Biol. 295: 613-615), other pairwise sequence comparison algorithms may be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP super families of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

It is within the knowledge of the skilled person to determine which alignment tool to use when corresponding amino acid positions must be identified. Therefore, it is contemplated that any available alignment tool that the skilled person find suitable may be used in the context of the present invention.

In describing the enzyme variants described herein, the nomenclature described below is adapted for ease of reference. The accepted IUPAC single letter or three letters amino acid abbreviations are employed. Amino acid positions are indicated with #₁, #₂, etc.

Substitutions: For an amino acid substitution, the following nomenclature is used: Original amino acid, position, substituted amino acid. Accordingly, the substitution of serine at position #₁ with tryptophan is designated as “Ser#₁ᵢTrp” or “S#₁W”. Multiple mutations are separated by addition marks (“+”) or by commas (“,”), e.g., “Ser#₁Trp +“Ser#₂Pro” or S#₁W, S#₂P, representing substitutions at positions #₁ and #₂ of serine (S) with tryptophan (W) and proline (P), respectively. If more than one amino acid may be substituted in a given position these are listed in brackets, such as [X] or {X}. Thus if both Trp and Lys according to the invention may be substituted instead of the amino acid occupying at position #₁ this is indicated as X#₁ {W, K} or X#₂ [W, K] where the X indicate that different enzymes may be parent e.g. such as a protease with SEQ ID NO 3 or a protease having at least 75% identity hereto. Thus, in some cases the variants are represented as #1 {W, K} or X#₂P indicating that the amino acids to be substituted vary depending on the parent.

Deletions: For an amino acid deletion, the following nomenclature is used: Original amino acid, position, *. Accordingly, the deletion of serine at position #₁ is designated as “Ser#₁*” or “S#₁*”.

Multiple deletions are separated by addition marks (“+”) or commas, e.g., “Ser#₁*+Ser#₂*” or “S#₁*, S#₂*”.

Insertions: The insertion of an additional amino acid residue such as e.g. a lysine after G#₁ may be indicated by: Gly#₁GlyLys or G#₁GK. Alternatively insertion of an additional amino acid residue such as lysine after G#₁ may be indicated by: *#ᵢaL. When more than one amino acid residue is inserted, such as e.g. a Lys, and Ala after #₁ this may be indicated as: Gly#₁GlyLysAla or G#₁GKA. In such cases, the inserted amino acid residue(s) may also be numbered by the addition of lower case letters to the position number of the amino acid residue preceding the inserted amino acid residue(s), in this example: *#₁aK *#₁bA.

Collectively, substitutions, deletions, and insertions may herein termed “modifications”. Thus, it is to be understood that any variant described herein comprises modifications, such as substitutions, deletions and/or insertions unless otherwise indicated by context.

Multiple modifications: Variants comprising multiple modifications are separated by addition marks (“+”), division marks (“/”), or by commas (“,”), e.g., “Ser#₁Trp+Ser#₂Pro” or “S#₁W, S#₂P” representing a substitution of serine at positions #₁ and #₂ with tryptophan and proline, respectively as described above.

Different modifications: Where different modifications can be introduced at a position, the different alterations are separated by a division (“/”), or by a comma (“,”), e.g., “Ser#₁Trp, Lys” or S#₁W, K represents a substitution of serine at position #₁ with tryptophan or lysine. Thus, “Ser#₁Trp, Lys+Ser#₂Asp” designates the following variants: “Ser#₁Trp+Ser#₂Pro”, “Ser∩₁Lys+Ser#₂Pro” or S#₁W, K+S#₂D.

### DETAILED DESCRIPTION OF THE INVENTION

In one aspect, the present invention relates to a detergent composition comprising (i) at least one protease variant comprising a substitution of one or more amino acids in the loop corresponding to positions 171, 173, 175, 179, or 180 of SEQ ID NO: 1, wherein the protease variant has a sequence identity of at least 75% but less than 100% to SEQ ID NO: 1, and wherein the protease variant has protease activity, and (ii) at least one alpha-amylase having the amino acid sequence of SEQ ID NO: 2, 3, 4, 5, 6, 7, 8, or 9, or a variant thereof having a sequence identity of at least 75% but less than 100% to SEQ ID NO: 2, 3, 4, 5, 6, 7, 8, or 9, and wherein said alpha-amylase has alpha-amylase activity. The protease variant of the detergent composition has been found to have improved stability in detergent compared to a protease having the identical amino acid sequence of said variant but not having the substitution(s) at one or more of said specified positions or compared to a protease with SEQ ID NO: 1. The amino acids corresponding to positions 171, 173, 175, 179, or 180 of SEQ ID NO: 1 form part of an active site loop corresponding to positions 170 to 180 of SEQ ID NO: 1, which defines parts of the substrate binding pocket. The protease variants of the detergent composition of the present invention containing one or more substitution(s) in the loop region 170-180 (SEQ ID NO: 1numbering), have been generated and were tested for stability in a model detergent as described in “Material and Methods” and the inventors demonstrate that one or more substitutions of one or more amino acid at a position corresponding to positions 171, 173, 175 or 179 of the mature polypeptide SEQ ID NO: 1 significantly improved the detergent stability compared to a protease having the identical amino acid sequence of said variant but not having a substitution at one or more of said specified positions or compared to a protease with SEQ ID NO: 1.

Preferably, the detergent composition according to the present invention constitutes a composition, which does not only show improved stability but also provides a composition having improved wash performance.

Thus, the invention relates to a detergent composition, wherein the at least one protease variant comprises a substitution of one or more amino acids in the loop corresponding to positions 171, 173, 175, 179, or 180 of SEQ ID NO: 1, wherein the protease variant has at least 75% sequence identity to the parent protease of SEQ ID NO: 1 (Bacillus sp. TY145), e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95, at least 96%, at least 97%, at least 98%, but less than 100% sequence identity to the parent protease of SEQ ID NO: 1.

It is to be understood that in the context of the present invention “a protease variant” or “the protease variant” means “at least one protease variant” unless contradicted by context, e.g. “the one protease variant”. Thus, the detergent composition according to the invention will in all embodiments comprise at least one protease variant. The same applies to the alpha-amylase or a variant thereof.

In one embodiment, the at least one protease variant comprises a substitution in at least one position corresponding to positions 173, 175, or 180 of SEQ ID NO: 1.

In a particular embodiment, the at least one protease variant comprises a substitution at one or more positions corresponding to positions 171, 173, 175, 179, or 180 of SEQ ID NO: 1, wherein the protease variant is a protease variant of a parent protease which has at least 70%, such as at least 71%, at least 72%, at least 73%, at least 74%, such as at least 75%, e.g., such as at least 76% at least 77% at least 78% at least 79% at least 80%, at least 81% at least 82% at least 83% at least 84% at least 85%, at least 86% at least 87% at least 88% at least 89%, at least 90%, at least 91%, at least 92%, at least 93%, at least 94%, at least 95%, at least 96%, at least 97%, at least 98%, at least 99% e. g. at least 99.1%, at least 99.2%, at least 99.3%, at least 99.4%, at least 99.5%, at least 99.6, or 100% sequence identity to SEQ ID NO: 1. In one particular embodiment, the at least one protease variant is a TY-145 (SEQ ID NO: 1) variant comprising a substitution of one or more amino acids in the loop corresponding to positions 171, 173, 175, 179, or 180 of SEQ ID NO: 1. In another embodiment, the at least one protease variant of the detergent composition according to the invention comprises a substitution at two, three, four or five positions corresponding to positions 171, 173, 175, 179, or 180 of SEQ ID NO: 1. A preferred embodiment concerns a detergent composition, wherein the at least one protease variant comprises a substitution of one or more amino acids in the loop corresponding to positions 171, 173, 175, 179, or 180 of SEQ ID NO: 1, wherein the variant has at least 70%, such as at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% at least 95% identity, at least 96%, at least 97%, at least 98%, or at least 99%, e. g. at least 99.1%, at least 99.2%, at least 99.3%, at least 99.4%, at least 99.5%, at least 99.6, but less than 100%, sequence identity to SEQ ID NO: 1.

Thus, in a further embodiment, the at least one protease variant comprises a substitution in at least one position corresponding to positions 171, 173, 175, 179, or 180, and wherein
a. the amino acid in the position corresponding to position 171 of SEQ ID NO: 1 is selected from the group consisting of W, K, E, D, and N; and/or
b. the amino acid in the position corresponding to position 173 of SEQ ID NO: 1 is P; and/or
c. the amino acid in the position corresponding to position 175 of SEQ ID NO: 1 is selected from the group consisting of A, V, and P; and/or
d. the amino acid in the position corresponding to position 179 of SEQ ID NO: 1 is selected from the group consisting of C, V, Q, S, T, E, H, K, M, N, Y, and A; and/or
e. the amino acid in the position corresponding to position 180 of SEQ ID NO: 1 is Y.

In one embodiment, the at least one protease variant comprises a substitution at position 171. In a preferred embodiment, the protease variant comprises a substitution at position 171 with W, K, E, D, or N. In another preferred embodiment, the at least one protease variant comprises a W at position 171. In yet another preferred embodiment, the at least one protease variant comprises the substitution S171W, wherein the parent protease is the mature polypeptide with SEQ ID NO: 1. In another preferred embodiment, the protease variant comprises an K at position 171. In yet another preferred embodiment, the protease variant comprises the substitution S171K, wherein the parent protease is the mature polypeptide with SEQ ID NO: 1. In another preferred embodiment, the at least one protease variant comprises an E at position 171. In yet another preferred embodiment, the at least one protease variant comprises the substitution S171E, wherein the parent protease is the mature polypeptide with SEQ ID NO: 1. In another preferred embodiment, the at least one protease variant comprises an N at position 171. In yet another preferred embodiment, the at least one protease variant comprises the substitution S171N, wherein the parent protease is the mature polypeptide with SEQ ID NO: 1.

In a further embodiment, the at least one protease variant comprises a substitution at position 171 with W, K, E, D, or N wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises a W at position 171. In yet another preferred embodiment, the at least one protease variant comprises the substitution S171W, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises a K at position 171. In yet another preferred embodiment, the at least one protease variant comprises the substitution S171K, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises an E at position 171. In yet another preferred embodiment, the at least one protease variant comprises the substitution S171E, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises an N at position 171. In yet another preferred embodiment, the at least one protease variant comprises the substitution S171N, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In an even further embodiment, the at least one protease variant comprises a substitution at position 171 with W, K, E or N, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%,e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises a W at position 171. In yet another preferred embodiment, the at least one protease variant comprises the substitution S171W, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%,e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises a K at position 171. In yet another preferred embodiment, the at least one protease variant comprises the substitution S171K, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%,e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises an E at position 171. In yet another preferred embodiment, the at least one protease variant comprises the substitution S171E, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said variant but not having the substitutions at one or more of said positions, when tested as described in the Examples. In another preferred embodiment, the at least one protease variant comprises an N at position 171. In yet another preferred embodiment, the at least one protease variant comprises the substitution S171N, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%,e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said variant but not having the substitutions at one or more of said positions, when tested as described in the Examples.

In one embodiment, the at least one protease variant comprises a substitution at position 173. In a preferred embodiment, the at least one protease variant comprises a P at position 173. In another preferred embodiment, the at least one protease variant comprises the substitution S173P, wherein the parent protease is a polypeptide of SEQ ID NO: 1. In yet another preferred embodiment, the at least one protease variant comprises the substitution S173P, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In yet another preferred embodiment, the at least one protease variant comprises the substitution S173P, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In one embodiment, the at least one protease variant comprises a substitution at position 175. In a preferred embodiment, the at least one protease variant comprises an A, V, or P at position 175. In another preferred embodiment, the at least one protease variant comprises an A at position 175. In yet another preferred embodiment, the at least one protease variant comprises the substitution S175A, wherein the parent protease is a polypeptide of SEQ ID NO: 1. In another preferred embodiment, the at least one protease variant comprises a V at position 175. In yet another preferred embodiment, the at least one protease variant comprises the substitution S175V, wherein the parent protease is the mature polypeptide with SEQ ID NO: 1. In another preferred embodiment, the at least one protease variant comprises a P at position 175. In yet another preferred embodiment, the at least one protease variant comprises the substitution S175P, wherein the parent protease is a polypeptide of SEQ ID NO: 1.

In a further embodiment, the at least one protease variant comprises an A, V, or P at position 175, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises an A at position 175. In yet another preferred embodiment, the at least one protease variant comprises the substitution S175A, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%,e. g. at least 99.1%, at least 99.2%, at least 99.3%, at least 99.4%, at least 99.5%, at least 99.6, but less than 100%. In another preferred embodiment, the at least one protease variant comprises a V at position 175. In yet another preferred embodiment, the at least one protease variant comprises the substitution S175V, wherein the protease variant has at least 70% sequence identity to SEQ ID NO: 11, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises a P at position 175. In yet another preferred embodiment, the at least one protease variant comprises the substitution S175P, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In an even further embodiment, the at least one protease variant comprises an A, V, or P at position 175, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises an A at position 175. In yet another preferred embodiment, the at least one protease variant comprises the substitution S175A, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises a V at position 175. In yet another preferred embodiment, the at least one protease variant comprises the substitution S175V, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises a P at position 175. In yet another preferred embodiment, the at least one protease variant comprises the substitution S175P, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In a further embodiment, the at least one protease variant comprises a C, V, Q, S, T, E, H, K, M, B, Y, or A at position 179. In a preferred embodiment, the at least one protease variant comprises a C, V, Q, S, T, E, H, K, M, B, Y, or A at position 179, and wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a further preferred embodiment, the at least one protease variant comprises a C, V, Q, S, T, E, H, K, M, B, Y, or A at position 179, and wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples. In another preferred embodiment, the at least one protease variant comprises a C at position 179. In yet another preferred embodiment, the at least one protease variant comprises the substitution G179C, wherein the parent protease is a polypeptide of SEQ ID NO: 1. In yet another preferred embodiment, the at least one protease variant comprises the substitution G179C, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, a g. at least 99.1%, at least 99.2%, at least 99.3%, at least 99.4%, at least 99.5%, at least 99.6, but less than 100%. In yet another preferred embodiment, the at least one protease variant comprises the substitution G179C, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 ora parent protease having the identical amino acid sequence of said protease variant but not having the substitutions at one or more of said positions, when tested as described in the Examples.

In another preferred embodiment, the at least one protease variant comprises a V at position 179. In yet another preferred embodiment, the at least one protease variant comprises the substitution G179V, wherein the parent protease is a polypeptide with SEQ ID NO: 1. In a further embodiment, the at least one protease variant comprises the substitution G179V, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises the substitution G179V, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises a Q at position 179. In yet another preferred embodiment, the at least one protease variant comprises the substitution G179Q, wherein the parent protease is a polypeptide of SEQ ID NO: 1. In a further embodiment, the at least one protease variant comprises the substitution G179Q, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179Q, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises an S at position 179. In yet another preferred embodiment, the at least one protease variant comprises the substitution G179S, wherein the parent protease is a polypeptide of SEQ ID NO: 1. In a further embodiment, the at least one protease variant comprises the substitution G179S, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, but less than 100%. In a another embodiment, the protease variant comprises the substitution G179S, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises a T at position 179. In yet another preferred embodiment, the at least one protease variant comprises the substitution G179T, wherein the parent protease is a polypeptide of SEQ ID NO: 1. In a further embodiment, the at least one protease variant comprises the substitution G179T, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another embodiment, the at least one protease variant comprises the substitution G179T, wherein the at least one protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises an E at position 179. In yet another preferred embodiment, the at least one protease variant comprises the substitution G179E, wherein the parent protease is a polypeptide of SEQ ID NO: 1. In a further embodiment, the at least one protease variant comprises the substitution G179E, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179E, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said variant but not having the substitutions at one or more of said positions, when tested as described in the Examples.

In another preferred embodiment, the at least one protease variant comprises an H at position 179. In yet another preferred embodiment, the at least one protease variant comprises the substitution G179H, wherein the parent protease is a polypeptide of SEQ ID NO: 1. In a further embodiment, the at least one protease variant comprises the substitution G179H, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179H, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises a K at position 179. In yet another preferred embodiment, the at least one protease variant comprises the substitution G179K, wherein the parent protease is a polypeptide with SEQ ID NO: 1. In a further embodiment, the at least one protease variant comprises the substitution G179K, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179K, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises an M at position 179. In yet another preferred embodiment, the at least one protease variant comprises the substitution G179M, wherein the parent protease is a polypeptide of SEQ ID NO: 1. In a further embodiment, the at least one protease variant comprises the substitution G179M, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, but less than 100%. In another embodiment, the at least one protease variant comprises a substitution at position 179 with M. In yet another preferred embodiment, the at least one protease variant comprises the substitution G179M, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said variant but not having the substitutions at one or more of said positions, when tested as described in the Examples.

In another preferred embodiment, the at least one protease variant comprises an N at position 179. In yet another preferred embodiment, the at least one protease variant comprises the substitution G179N, wherein the parent protease is a polypeptide of SEQ ID NO: 1. In a further embodiment, the at least one protease variant comprises the substitution G179N, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a further embodiment, the at least one protease variant comprises the substitution G179N, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In another preferred embodiment, the at least one protease variant comprises an A at position 179. In yet another preferred embodiment, the at least one protease variant comprises the substitution G179A, wherein the parent protease is a polypeptide with SEQ ID NO: 1. In a further embodiment, the at least one protease variant comprises the substitution G179A, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179A, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said variant but not having the substitutions at one or more of said positions, when tested as described in the Examples.

In another preferred embodiment, the at least one protease variant comprises a Y at position 179. In yet another preferred embodiment, the at least one protease variant comprises the substitution G179Y, wherein the parent protease is a polypeptide of SEQ ID NO: 1. In a further embodiment, the at least one protease variant comprises the substitution G179Y, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In a another embodiment, the at least one protease variant comprises the substitution G179Y, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in the Examples.

In one embodiment, the at least one protease variant comprises a Y at position 180. In a preferred aspect, the at least one protease variant comprises the substitution F180Y, wherein the parent protease is a polypeptide of SEQ ID NO: 1. In a further embodiment, the at least one protease variant comprises the substitution F180Y, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In another preferred embodiment, the at least one protease variant comprises the substitution F180Y, wherein the protease variant has at least 70% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%, and having an increased stability relative to a protease of SEQ ID NO: 1 or a parent protease having the identical amino acid sequence of said protease variant but not having the substitutions at one or more of said positions, when tested as described in Examples.

In one embodiment, the at least one alpha-amylase is a variant of a parent alpha-amylase of SEQ ID NO: 2, 3, 4, 5, 6, 7, 8, or 9, and wherein the alpha-amylase variant has alpha-amylase activity. Thus, in one embodiment, the at least one alpha-amylase is a variant of a parent alpha-amylase of SEQ ID NO: 2. In a preferred embodiment, the at least one alpha-amylase variant comprises a deletion and/or a substitution at two or more positions corresponding to positions 181, 182, 183, and 184 of SEQ ID NO: 2, wherein the alpha-amylase variant has at least 75% sequence identity to SEQ ID NO: 2, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%. In one embodiment, the at least one alpha-amylase variant comprises one or more modifications in the following positions: 140, 195, 206, 243, 260, and 476 of SEQ ID NO: 2, wherein the at least one alpha-amylase variant has at least 75% sequence identity to SEQ ID NO: 2, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 1. In a preferred embodiment, the at least one alpha-amylase variant comprises one or more modifications in the following positions: 9, 118, 149, 182, 186, 195, 202, 257, 295, 299, 320, 323, 339, 345, and 458 of SEQ ID NO: 1, wherein the at least one alpha-amylase variant has at least 75% sequence identity to SEQ ID NO: 1, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 4. In one preferred embodiment, the at least one alpha-amylase variant comprises one or more modifications in the following positions: 180, 181, 243, and 475 of SEQ ID NO: 4, wherein the at least one alpha-amylase variant has at least 75% sequence identity to SEQ ID NO: 4, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 5. In one preferred embodiment, the at least one alpha-amylase variant comprises one or more modifications in the following positions: 178, 179, 187, 203, 458, 459, 460, and 476 of SEQ ID NO: 5, wherein the at least one alpha-amylase variant has at least 75% sequence identity to SEQ ID NO: 5, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 6. In one preferred embodiment, the at least one alpha-amylase variant comprises a modification in position 202 of SEQ ID NO: 6, wherein the at least one alpha-amylase variant has at least 75% sequence identity to SEQ ID NO: 6, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 7. In a preferred embodiment, the at least one alpha-amylase variant comprises one or more modifications in the following positions: 405, 421, 422, and 428 of SEQ ID NO: 7, wherein the at least one alpha-amylase variant has at least 75% sequence identity to SEQ ID NO: 7, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 8. In one embodiment, the at least one alpha-amylase variant has at least 75% sequence identity to SEQ ID NO: 8, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In another embodiment, the at least one alpha-amylase is a variant of a parent alpha-amylase of SEQ ID NO: 9. In one preferred embodiment, the at least one alpha-amylase variant comprises one or more modifications in the following positions: 48, 49, 107, 156, 181, 190, 209, and 264 of SEQ ID NO: 9, wherein the at least one alpha-amylase variant has at least 75% sequence identity to SEQ ID NO: 9, such as at least 71%, at least 72%, at least 73%, at least 74%, at least 75%, at least 80%, at least 85%, at least 90%, at least 95%, at least 96%, at least 97%, at least 98%, or at least 99%, e. g. at least 99.1%, at least 99.2%, at least 99.3%, at least 99.4%, at least 99.5%, at least 99.6, but less than 100%.

In one embodiment, the detergent composition comprises a protease variant comprising a W, K, E, D, or N at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a W, K, E, D, or N at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a W, K, E, D, or N at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a W, K, E, D, or N at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a W, K, E, D, or N at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a W, K, E, D, and N at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a W, K, E, D, and N at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a W, K, E, D, or N at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a W, K, E, D, or N at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a W at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a W at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a W at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a W at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a W at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a W at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a W at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a W at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a W at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a K at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a K at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a K at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a K at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a K at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a K at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a K at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a K at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a K at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a E at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a E at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a E at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a E at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a E at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a E at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a E at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a E at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a E at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a D at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a D at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a D at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a D at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a D at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a D at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a D at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a D at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a D at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a N at position 171 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a N at position 171 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a N at position 171 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a N at position 171 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a N at position 171 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a N at position 171 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a N at position 171 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a N at position 171 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a N at position 171 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a P at position 173 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a P at position 173 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a P at position 173 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a P at position 173 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a P at position 173 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a P at position 173 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a P at position 173 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a P at position 173 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a P at position 173 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an A, V, or P at position 175 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A, V, or P at position 175 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising an A, V, or P at position 175 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A, V, or P at position 175 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an A, V, or P at position 175 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an A, V, or P at position 175 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an A, V, or P at position 175 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an A, V, or P at position 175 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an A, V, or P at position 175 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an A at position 175 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A at position 175 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising an A at position 175 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A at position 175 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an A at position 175 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an A at position 175 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an A at position 175 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an A at position 175 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an A at position 175 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a V at position 175 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a V at position 175 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a V at position 175 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a V at position 175 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a V at position 175 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a V at position 175 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a V at position 175 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a V at position 175 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a V at position 175 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a P at position 175 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a P at position 175 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a P at position 175 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a P at position 175 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a P at position 175 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a P at position 175 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a P at position 175 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a P at position 175 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a P at position 175 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a C, V, Q, S, T, E, H, K, M, N, Y or A at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a C, V, Q, S, T, E, H, K, M, N, Y or A at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a C, V, Q, S, T, E, H, K, M, N, Y or A at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a C, V, Q, S, T, E, H, K, M, N, Y or A at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a C at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a C at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a C at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a C at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a C at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a C at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a C at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a C at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a C at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a V at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a V at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 1, and wherein the protease and the alpha-amylase variants each have at least 75% but less than 100% sequence identity to SEQ ID NO: 1 and 3, respectively.

In one embodiment, the detergent composition comprises a protease variant comprising a V at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a V at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a V at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a V at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a V at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a V at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a V at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a Q at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Q at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a Q at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Q at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a Q at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a Q at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a Q at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a Q at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a Q at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a S at position 179 of SEQ ID NO: 1, and an alpha-amylase variant comprising a deletion and/or a substitution at two or more positions corresponding to positions 181, 182, 183, and 184 of SEQ ID NO: 42 and wherein the protease and the alpha-amylase variants each have at least 75% but less than 100% sequence identity to SEQ ID NO: 1 and 2, respectively.

In one embodiment, the detergent composition comprises a protease variant comprising a S at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a S at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a S at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a S at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a S at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a S at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a S at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a S at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a T at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a T at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a T at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a T at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a T at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a T at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a T at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a T at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a T at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an E at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an E at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an E at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an E at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an E at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an E at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an E at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an Eat position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an E at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an H at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an H at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an H at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an H at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an H at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an H at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an H at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an H at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an H at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an K at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an K at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an K at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an K at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an K at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an K at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an K at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an K at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an K at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an M at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an M at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an M at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an M at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an M at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an M at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an M at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an M at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an M at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an N at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an N at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an N at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an N at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an N at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an N at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an N at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an N at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an N at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a Y at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Y at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a Y at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Y at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a Y at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a Y at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a Y at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a Y at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a Y at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising an A at position 179 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A at position 179 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising an A at position 179 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising an A at position 179 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising an A at position 179 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising an A at position 179 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising an A at position 179 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising an A at position 179 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising an A at position 179 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a Y at position 180 of SEQ ID NO: 1, and an alpha-amylase variant comprising a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Y at position 180 of SEQ ID NO: 1, and an alpha-amylase variant comprising one or more modifications in the following positions: 9, 118, 149, 182, 186, 195, 202, 257, 295, 299, 320, 323, 339, 345, and 458 of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a Y at position 180 of SEQ ID NO: 1, and an alpha-amylase variant comprising one or more modifications in the following positions: 140, 195, 206, 243, 260, and 476 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a Y at position 180 of SEQ ID NO: 1, and an alpha-amylase variant comprising one or more modifications in the following positions: 180, 181, 243, and 475 of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising a Y at position 180 of SEQ ID NO: 1, and an alpha-amylase variant comprising one or more modifications in the following positions: 178, 179, 187, 203, 458, 459, 460, and 476 of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising a Y at position 180 of SEQ ID NO: 1, and an alpha-amylase variant comprising a modification in position 202 of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a Y at position 180 of SEQ ID NO: 1, and an alpha-amylase variant comprising one or more modifications in the following positions: 405, 421, 422, and 428 of SEQ ID NO: 7, and wherein the protease and the alpha-amylase variants each have at least 75% but less than 100% sequence identity to SEQ ID NO: 1 and 7, respectively.

In one embodiment, the detergent composition comprises a protease variant comprising a Y at position 180 of SEQ ID NO: 1, and an alpha-amylase variant of a parent alpha-amylase of SEQ ID NO: 8, and wherein the protease and the alpha-amylase variants each have at least 75% sequence identity to SEQ ID NO: 1 and 8, respectively.

In one embodiment, the detergent composition comprises a protease variant comprising a Y at position 180 of SEQ ID NO: 1, and an alpha-amylase variant comprising one or more modifications in the following positions: 48, 49, 107, 156, 181, 190, 209, and 264 of SEQ ID NO: 9, and wherein the protease and the alpha-amylase variants each have at least 75% but less than 100% sequence identity to SEQ ID NO: 1 and 9, respectively.

In a particular embodiment, the at least one alpha-amylase variant is selected from the group consisting of:

a. an alpha-amylase comprising a deletion in the positions corresponding to D183 and G184 of SEQ ID NO: 2;

b. an alpha-amylase comprising the modifications R118K, D183*, G184*, N195F, R320K, and R458K of SEQ ID NO: 3;

c. an alpha-amylase comprising the modifications M9L, R118K, G149A, G182T, G186A, D183*, G184*, N195F, M202L, T257I, Y295F, N299Y, R320K, M323T, A339S, E345R, and R458K of SEQ ID NO: 3;

d. an alpha-amylase comprising the modifications D183*, G184*, W140Y, N195F, and 1206Y of SEQ ID NO: 2;

e. an alpha-amylase comprising the modifications R180*, S181*, S243Q, G475K, and CBM* of SEQ ID NO: 4;

f. an alpha-amylase comprising the modifications R178*, G179*, E187P, 1203Y, R458N,T459S, D460T, and G476K of SEQ ID NO: 5;

g. an alpha-amylase comprising the substitution M202L of SEQ ID NO: 6;

h. an alpha-amylase comprising the substitution M197T of SEQ ID NO: 9;

i. an alpha-amylase comprising the substitutions H156Y, A181T, N190F, A209V, and Q264S of SEQ ID NO: 9; and j. an alpha-amylase comprising the substitutions G48A, T491, G107A, H156Y, A181T, N190F, L201F, A209V, and Q264S of SEQ ID NO: 9.

In one embodiment, the detergent composition comprises a protease variant comprising or consisting of a W, K, E, D, or N at position 171 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a W, K, E, D, or N at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a W, K, E, D, or N at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a W, K, E, D, or N at position 171 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a W, K, E, D, or N at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a W, K, E, D, or N at position 171 of SEQ ID NO: 1, and an alpha-amylase variant comprising or consisting of the modifications R178*, G179*, E187P, 1203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a W, K, E, D, and N at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a W, K, E, D, and N at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a W, K, E, D, or N at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a W, K, E, D, or N at position 171 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a W at position 171 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a W at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a W at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a W at position 171 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a W at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a W at position 171 of SEQ ID NO: 1, and an alpha-amylase variant comprising or consisting of the modifications R178*, G179*, E187P, 1203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a W at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a W at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a W at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a W at position 171 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a K at position 171 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a K at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a K at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a K at position 171 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a K at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a K at position 171 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a K at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a K at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a K at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a K at position 171 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an E at position 171 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an E at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an E at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an E at position 171 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an E at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an E at position 171 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an E at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an E at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an E at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an E at position 171 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a N at position 171 of SEQ ID NO: 1, and an alpha-amylase variant comprising or consisting of a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a N at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a N at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a N at position 171 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a N at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a N at position 171 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a N at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a N at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a N at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a N at position 171 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a D at position 171 of SEQ ID NO: 1, and an alpha-amylase variant comprising or consisting of a deletion and/or a substitution at two or more positions corresponding to positions 181, 182, 183, and 184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a D at position 171 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a D at position 171 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a D at position 171 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a D at position 171 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a D at position 171 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a D at position 171 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a D at position 171 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a D at position 171 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a D at position 171 of SEQ ID NO: 1, and an alpha-amylase variant comprising or consisting of the substitutions G48A, T491, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A, V, or P at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A, V, or P at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an A, V, or P at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an A, V, or P at position 175 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A, V, or P at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an A, V, or P at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an A, V, or P at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an A, V, or P at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A, V, or P at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an A, V, or P at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an A at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an A at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an A at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an A at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an A at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an A at position 175 of SEQ ID NO: 1, and an alpha-amylase variant comprising or consisting of the substitutions G48A, T491, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a or consisting of V at position 175 of SEQ ID NO: 1SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a V at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a V at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variant each have at least 75% but less than 100% sequence identity to SEQ ID NO: 1 and 3, respectively.

In one embodiment, the detergent composition comprises a protease variant comprising or consisting of a V at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a V at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a V at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a V at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a V at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a V at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a V at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 175 of SEQ ID NO: 1SEQ ID NO: 1, and an alpha-amylase variant comprising or consisting of a deletion in the positions corresponding to D183 and G184 of SEQ ID NO: 4, and wherein the protease and the alpha-amylase variants each have at least 75% but less than 100% sequence identity to SEQ ID NO: 1SEQ ID NO: 1 and 4, respectively.

In one embodiment, the detergent composition comprises a protease variant comprising or consisting of a P at position 175 of SEQ ID NO: 1SEQ ID NO: 1, and an alpha-amylase variant comprising or consisting of the modifications R118K, D183*, G184*, N195F, R320K, and R458K of SEQ ID NO: 5, and wherein the protease and the alpha-amylase variants each have at least 75% but less than 100% sequence identity to SEQ ID NO: 1SEQ ID NO: 1 and 5, respectively.

In one embodiment, the detergent composition comprises a protease variant comprising or consisting of a P at position 175 of SEQ ID NO: 1SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 5, and wherein the protease and the alpha-amylase variants each have at least 75% but less than 100% sequence identity to SEQ ID NO: 1SEQ ID NO: 1 and 5, respectively.

In one embodiment, the detergent composition comprises a protease variant comprising or consisting of a P at position 175 of SEQ ID NO: 1SEQ ID NO: 1, and an alpha-amylase variant comprising or consisting of the modifications D183*, G184*, W140Y, N195F, and I206Y of SEQ ID NO: 4, and wherein the protease and the alpha-amylase variants each have at least 75% but less than 100% sequence identity to SEQ ID NO: 1SEQ ID NO: 1 and 4, respectively.

In one embodiment, the detergent composition comprises a protease variant comprising or consisting of a P at position 175 of SEQ ID NO: 1SEQ ID NO: 1, and an alpha-amylase variant comprising or consisting of the modifications R180*, S181*, S243Q, G475K, and CBM* of SEQ ID NO: 6, and wherein the protease and the alpha-amylase variants each have at least 75% but less than 100% sequence identity to SEQ ID NO: 1SEQ ID NO: 1 and 6, respectively.

In one embodiment, the detergent composition comprises a protease variant comprising or consisting of a P at position 175 of SEQ ID NO: 1SEQ ID NO: 1, and an alpha-amylase variant comprising or consisting of the modifications R178*, G179*, E187P, I203Y, R458N, T459S, D460T, and G476K of SEQ ID NO: 7, and wherein the protease and the alpha-amylase variants each have at least 75% but less than 100% sequence identity to SEQ ID NO: 1SEQ ID NO: 1 and 7, respectively.

In one embodiment, the detergent composition comprises a protease variant comprising or consisting of a P at position 175 of SEQ ID NO: 1SEQ ID NO: 1, and an alpha-amylase variant comprising or consisting of the substitution M202L of SEQ ID NO: 8, and wherein the protease and the alpha-amylase variants each have at least 75% but less than 100% sequence identity to SEQ ID NO: 1SEQ ID NO: 1 and 8, respectively.

In one embodiment, the detergent composition comprises a protease variant comprising or consisting of a P at position 175 of SEQ ID NO: 1SEQ ID NO: 1, and an alpha-amylase variant comprising or consisting of the substitution M197T of SEQ ID NO: 11, and wherein the protease and the alpha-amylase variants each have at least 75% but less than 100% sequence identity to SEQ ID NO: 1SEQ ID NO: 1 and 11, respectively.

In one embodiment, the detergent composition comprises a protease variant comprising or consisting of a P at position 175 of SEQ ID NO: 1SEQ ID NO: 1, and an alpha-amylase variant comprising or consisting of the substitutions H156Y, A181T, N190F, A209V, and Q264S of SEQ ID NO: 11, and wherein the protease and the alpha-amylase variants each have at least 75% sequence identity to SEQ ID NO: 1SEQ ID NO: 1 and 11, respectively.

In one embodiment, the detergent composition comprises a protease variant comprising or consisting of a P at position 175 of SEQ ID NO: 1SEQ ID NO: 1, and an alpha-amylase variant comprising or consisting of the substitutions G48A, T49I, G107A, H156Y, A181T, N190F, L201F, A209V, and Q264S of SEQ ID NO: 11, and wherein the protease and the alpha-amylase variants each have at least 75% but less than 100% sequence identity to SEQ ID NO: 1SEQ ID NO: 1 and 11, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of a deletion in the positions corresponding to D183 and G184 of SEQ ID NO: 4, and wherein the protease and the alpha-amylase variants each have at least 75% but less than 100% sequence identity to SEQ ID NO: 1SEQ ID NO: 1 and 4, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the modifications R118K, D183*, G184*, N195F, R320K, and R458K of SEQ ID NO: 5, and wherein the protease and the alpha-amylase variants each have at least 75% but less than 100% sequence identity to SEQ ID NO: 1SEQ ID NO: 1 and 5, respectively.

In one embodiment, the detergent composition comprises a protease variant comprising or consisting of a C, V, Q, S, T, E; H, K, M, N, Y, or A at position 179 of SEQ ID NO: 1SEQ ID NO: 1, and an alpha-amylase variant comprising or consisting of the modifications M9L, R118K, G149A, G182T, G186A, D183*, G184*, N195F, M202L, T2571, Y295F, N299Y, R320K, M323T, A339S, E345R, and R458K of SEQ ID NO: 5, and wherein the protease and the alpha-amylase variants each have at least 75% but less than 100% sequence identity to SEQ ID NO: 1SEQ ID NO: 1 and 5, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the modifications D183*, G184*, W140Y, N195F, and I206Y of SEQ ID NO: 4, and wherein the protease and the alpha-amylase variants each have at least 75% but less than 100% sequence identity to SEQ ID NO: 1SEQ ID NO: 1 and 4, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the modifications R180*, S181*, S243Q, G475K, and CBM* of SEQ ID NO: 6, and wherein the protease and the alpha-amylase variants each have at least 75% but less than 100% sequence identity to SEQ ID NO: 1SEQ ID NO: 1 and 6, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the modifications R178*, G179*, E187P, I203Y, R458N, T459S, D460T, and G476K of SEQ ID NO: 7, and wherein the protease and the alpha-amylase variants each have at least 75% but less than 100% sequence identity to SEQ ID NO: 1SEQ ID NO: 1 and 7, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the substitution M202L of SEQ ID NO: 8, and wherein the protease and the alpha-amylase variants each have at least 75% but less than 100% sequence identity to SEQ ID NO: 1SEQ ID NO: 1 and 8, respectively.

In one embodiment, the detergent composition comprises a protease variant comprising or consisting of a C, V, Q, S, T, E, H, K, M, N, Y or A at position 179 of SEQ ID NO: 1SEQ ID NO: 1, and an alpha-amylase variant comprising or consisting of the substitution M197T of SEQ ID NO: 11, and wherein the protease and the alpha-amylase variants each have at least 75% but less than 100% sequence identity to SEQ ID NO: 1SEQ ID NO: 1 and 11, respectively.

In one embodiment, the detergent composition comprises a protease variant comprising or consisting of a C, V, Q, S, T, E, H, K, M, N, Y or A at position 179 of SEQ ID NO: 1, and an alpha-amylase variant comprising or consisting of the substitutions H156Y, A181T, N190F, A209V, and Q264S of SEQ ID NO: 11, and wherein the protease and the alpha-amylase variants each have at least 75% sequence identity to SEQ ID NO: 1 and 11, respectively.

In one embodiment, the detergent composition comprises a protease variant comprising or consisting of a C, V, Q, S, T, E, H, K, M, N, Y or A at position 179 of SEQ ID NO: 1, and an alpha-amylase variant comprising or consisting of the substitutions G48A, T491, G107A, H156Y, A181T, N190F, L201F, A209V, and Q264S of SEQ ID NO: 11, and wherein the protease and the alpha-amylase variants each have at least 75% but less than 100% sequence identity to SEQ ID NO: 1 and 11, respectively.

In one embodiment, the detergent composition comprises a protease variant comprising or consisting of a C at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 4, respectively.

In one embodiment, the detergent composition comprises a protease variant comprising or consisting of a C at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a C at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a C at position 179 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a C at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a C at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a C at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a C at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a C at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a C at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a V at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a V at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a V at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a V at position 179 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a V at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a V at position 179 of SEQ ID NO: 1, and an alpha-amylase variant comprising or consisting of the modifications R178*, G179*, E187P, 1203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a V at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a V at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a V at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a V at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Q at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Q at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a Q at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a Q at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Q at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a Q at position 179 of SEQ ID NO: 1, and an alpha-amylase variant comprising or consisting of the modifications R178*, G179*, E187P, 1203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a Q at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising a Q at position 179 of SEQ ID NO: 1, and an alpha-amylase variant comprising the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Q at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a Q at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an S at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an S at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an S at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an S at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an S at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an S at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an S at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an S at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an S at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an S at position 179 of SEQ ID NO: 1, and an alpha-amylase variant comprising or consisting of the substitutions G48A, T491, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a T at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a T at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a T at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a T at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a T at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a T at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a T at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a T at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a T at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a T at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an E at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an E at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an E at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an E at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an E at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an E at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an E at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an E at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an E at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an E at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an H at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an H at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an H at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an H at position 179 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an H at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an H at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an H at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an H at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an H at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an H at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising a or consisting of K at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a or consisting of K at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a or consisting of K at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising a or consisting of K at position 179 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising a or consisting of K at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a K at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a K at position 179 of SEQ ID NO: 1, and an alpha-amylase variant comprising or consisting of the substitution M202L of SEQ ID NO: 8, and wherein the protease and the alpha-amylase variants each have at least 75% but less than 100% sequence identity to SEQ ID NO: 1 and 6, respectively.

In one embodiment, the detergent composition comprises a protease variant comprising or consisting of a K at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a K at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a K at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an M at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an M at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an M at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an M at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an M at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an M at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an M at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an M at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an M at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an M at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an N at position 179 of SEQ ID NO: and an alpha-amylase comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an N at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an N at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an N at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an N at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an N at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Y at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Y at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a Y at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a Y at position 179 of SEQ ID NO: 1, and an alpha-amylase variant comprising or consisting of the modifications D183*, G184*, W140Y, N195F, and 1206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Y at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a Y at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a Y at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a Y at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Y at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a Y at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an A at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of an A at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an A at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an A at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an A at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an A at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an A at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an A at position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Y at position 180 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Y at position 180 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a Y at position 180 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a Y at position 180 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a Y at position 180 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a Y at position 180 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a Y at position 180 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a Y at position 180 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a Y at position 180 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a Y at position 180 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In a particular embodiment, the detergent composition comprises further one or more additional enzymes. In a preferred embodiment, the detergent composition further comprises one or more lipases having the amino acid sequence of SEQ ID NO: 10, or a variant thereof having a sequence identity of at least 75% but less than 100% to SEQ ID NO: 10, and wherein the lipase variant has lipase activity.

In another embodiment, the protease variant comprises substitutions at positions corresponding to positions 171 and 173, such as those described above.

In another embodiment, the protease variant comprises substitutions at positions corresponding to positions 171 and 175, such as those described above.

In another embodiment, the protease variant comprises substitutions at positions corresponding to positions 171 and 179, such as those described above.

In another embodiment, the protease variant comprises substitutions at positions corresponding to positions 171 and 180, such as those described above.

In another embodiment, the protease variant comprises substitutions at positions corresponding to positions 173 and 175, such as those described above.

In another embodiment, the protease variant comprises substitutions at positions corresponding to positions 173 and 179, such as those described above.

In another embodiment, the protease variant comprises substitutions at positions corresponding to positions 173 and 180, such as those described above.

In another embodiment, the protease variant comprises substitutions at positions corresponding to positions 175 and 179, such as those described above.

In another embodiment, the protease variant comprises substitutions at positions corresponding to positions 175 and 180, such as those described above.

In another embodiment, the protease variant comprises substitutions at positions corresponding to positions 171, 173, and 175, such as those described above.

In another embodiment, the protease variant comprises substitutions at positions corresponding to positions 171, 173, and 179, such as those described above.

In another embodiment, the protease variant comprises substitutions at positions corresponding to positions 171, 173, and 180, such as those described above.

In another embodiment, the protease variant comprises substitutions at positions corresponding to positions 171, 175, and 179, such as those described above.

In another embodiment, the protease variant comprises substitutions at positions corresponding to positions 171, 175, and 180, such as those described above.

In another embodiment, the protease variant comprises substitutions at positions corresponding to positions 171, 179, and 180, such as those described above.

In another embodiment, the protease variant comprises substitutions at positions corresponding to positions 173, 175, and 179, such as those described above.

In another embodiment, the protease variant comprises substitutions at positions corresponding to positions 173, 175, and 180, such as those described above.

In another embodiment, the protease variant comprises substitutions at positions corresponding to positions 175, 179, and 180, such as those described above.

In another embodiment, the protease variant comprises substitutions at positions corresponding to positions 171, 173, 175, and 179, such as those described above.

In another embodiment, the protease variant comprises substitutions at positions corresponding to positions 171, 173, 175, and 180, such as those described above.

In another embodiment, the protease variant comprises substitutions at positions corresponding to positions 173, 175, 179 and 180, such as those described above.

In another embodiment, the protease variant comprises substitutions at positions corresponding to positions 171, 173, 175, 179 and 180, such as those described above.

In one embodiment, the number of modifications in the protease, alpha-amylase and/or lipase variants individually is 1 to 30, e.g.1 to 20, 1 to 10 and 1 to 5, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 modifications.

In one embodiment, the number of modifications in the protease, alpha-amylase and/or lipase variants individually is 1 to 20, e.g.1 to 10 and 1 to 5, such as 1, 2, 3, 4, 5, 6, 7, 8, 9, or 10 modifications.

In one embodiment, the detergent composition comprises a protease variant comprising or consisting of a P at position 173 and an A at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 and an A at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173 and an A at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a P at position 173 and an A at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 and an A at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173 and an A at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173 and an A at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173 and an A at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173 and an A at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173 and an A at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173 and a P at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 and a P at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173 and a P at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a P at position 173 and a P at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173 and a P at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173 and a P at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173 and a P at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173 and a P at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173 and a P at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173 and a P at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a P at position 173, a P at position 175, and a Y at position 180 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173, a P at position 175, and a Y at position 180 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a D at position 171, a P at position 173, and a P at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a D at position 171, a P at position 173, and a P at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a D at position 171, a P at position 173, and a P at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a K at position 171, a P at position 173, and a P at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a K at position 171, a P at position 173, and a P at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a K at position 171, a P at position 173, and a P at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 171, a P at position 173, and a P at position 175 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of an N at position 171, a P at position 173, and a P at position 175 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an N at position 171, a P at position 173, and a P at position 175 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and an M at position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a P at position 173, a P at position 175, and an M at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173, a P at position 175, and an M at position 179 of SEQ ID NO: 1, and an alpha-amylase variant comprising or consisting of the substitutions G48A, T491, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of a P at position 173, a P at position 175, and a T at position 179 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of a P at position 173, a P at position 175, and a T at position 179 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of a P at position 173, a P at position 175, and a T at position 179 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of a P at position 173, a P at position 175, and a T at position 179 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of a P at position 173, a P at position 175, and a Tat position 179 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of a deletion in the positions corresponding to D183 and G184 of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the modifications R118K, D183*, G184*, N195F, R320K, and R458K of SEQ ID NO: 3, and wherein the protease and the alpha-amylase variants each have at least 75% but less than 100% sequence identity to SEQ ID NO: 1 and 3, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the modifications M9L, R118K, G149A, G182T, G186A, D183*, G184*, N195F, M202L, T257I, Y295F, N299Y, R320K, M323T, A339S, E345R, and R458K of SEQ ID NO: 3, and wherein the protease and the alpha-amylase variants each have at least 75% but less than 100% sequence identity to SEQ ID NO: 1 and 3, respectively,

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the modifications D183*, G184*, W140Y, N195F, and I206Y of SEQ ID NO: 2, and wherein the protease and the alpha-amylase variants each have at least 75% but less than 100% sequence identity to SEQ ID NO: 1 and 2, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the modifications R180*, S181*, S243Q, G475K, and CBM* of SEQ ID NO: 4, and wherein the protease and the alpha-amylase variants each have at least 75% but less than 100% sequence identity to SEQ ID NO: 1 and 4, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the modifications R178*, G179*, E187P, I203Y, R458N, T459S, D460T, and G476K of SEQ ID NO: 5, and wherein the protease and the alpha-amylase variants each have at least 75% but less than 100% sequence identity to SEQ ID NO: 1 and 5, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the substitution M202L of SEQ ID NO: 6, and wherein the protease and the alpha-amylase variants each have at least 75% but less than 100% sequence identity to SEQ ID NO: 1 and 6, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the substitution M197T of SEQ ID NO: 9, and wherein the protease and the alpha-amylase variants each have at least 75% but less than 100% sequence identity to SEQ ID NO: 1 and 9, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the substitutions H156Y, A181T, N190F, A209V, and Q264S of SEQ ID NO: 9, and wherein the protease and the alpha-amylase variants each have at least 75% sequence identity to SEQ ID NO: 1 and 9, respectively.

In one embodiment, the detergent composition comprises a protease variant comprising or consisting of an N at position 171, a P at position 173, a P at position 175, and a Y at position 180 of SEQ ID NO: 1, and an alpha-amylase variant comprising or consisting of the substitutions G48A, T49I, G107A, H156Y, A181T, N190F, L201F, A209V, and Q264S of SEQ ID NO: 9, and wherein the protease and the alpha-amylase variants each have at least 75% but less than 100% sequence identity to SEQ ID NO: 1 and 9, respectively.

The protease variant may further comprise a substitution at one or more (several) other positions. For example, the variants may comprise an alteration at a position corresponding to positions selected from the group consisting of 39, 40, 70, 74, 81, 102, 121, 132, 137, 144, 155, 159, 162, 174, 176; 177, 241, 247, 256, 274, 286, 297 of SEQ ID NO 3. In one embodiment the variant of the invention comprises one of the following substitutions: Y39D; T40{D,P}; Q7ON; T74M; L81{F,H,V}; A102T; I121{V,T}; G132 {I, E}; I137{M;E}; 5144{Q,R}; D155N; G159S; V162R; G174{S,T}; N176G; T177S; T241P; I247M; H256F;S274I; V286Q; T297P wherein each position correspond to the positions of SEQ ID NO: 1.

In one embodiment, the detergent composition of the invention comprises a protease variant which comprises a substitution at one or more (e.g., several) positions corresponding to positions 171, 173, 175 or 179 of SEQ ID NO: 1. In a particular embodiment, the protease variant comprises a substitution at two positions corresponding to any of positions 171, 173, 175, 179 and 180 of SEQ ID NO: 1. In another embodiment, the protease variant comprises a substitution at three positions corresponding to any of positions 171, 173, 175, 179 and 180 of SEQ ID NO: 1. In another embodiment, the protease variant comprises a substitution at four positions corresponding to any of positions 171, 173, 175, 179 and 180 of SEQ ID NO: 1. In another embodiment, the protease variant comprises a substitution at each position corresponding to positions 171, 173, 175, 179 and 180 of SEQ ID NO: 1.

The protease, alpha-amylase and lipase and variants thereof may further comprise one or more additional alterations at one or more (e.g., several) other positions. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Asn/Gln, Thr/Ser, Ala/Gly, Ala/Thr, Ser/Asn, Ala/Val, Ser/Gly, Tyr/Phe, Ala/Pro, Lys/Arg, Asp/Asn, Glu/Gln, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

In one embodiment, the protease variants comprise any of the following substitutions compared to SEQ ID NO 3:

S171N S175P
I121V S175P
L81V S175P
A102T S175P
I137E S175P
S175A G179S
I121V S175A
L81F S175A
L81H S175A
L81V S175A
A102T S175A
I137E S175A
S144Q S175P
S144Q S175A
S144R G179Q
I121T S175P
S144R S171N
S173P S274I
I137M S173P
S171N S173P
L81F S173P
L81H S173P
A102T S173P
S144Q S173P
S144R F180Y
I137E S173P
S173P S175P F180Y
S173Y G174S S175A F180Y
S173P G174T S175V T177S F180Y
S173P G174K S175P N176G T177S F180Y
S173P T241P
G179S F180Y G183A A187V
S173Y G174S S175A F180Y T241P
D155N G159S S173Y G174S S175A F180Y
S173Y G174S S175A F180Y S274I
S173Y G174S S175A F180Y V286Q
T40D S173Y G174S S175A F180Y
V162R S173P G174T S175V T177S F180Y
I121V S173P G174T S175V T177S F180Y
L81V S173P G174T S175V T177S F180Y
A102T S173P G174T S175V T177S F180Y
S173P G174T S175V T177S F180Y T241P
I137E S173P G174T S175V T177S F180Y
S144Q S173P G174T S175V T177S F180Y
Q70N S173P G174T S175V T177S F180Y
D155N G159S S173P G174T S175V T177S F180Y
S173P G174T S175V T177S F180Y S274I
S173P G174T S175V T177S F180Y V286Q
T40D S173P G174T S175V T177S F180Y
S171N S173P G174T S175V T177S F180Y
V162R S173P G174K S175P N176G T177S F180Y
I121V S173P G174K S175P N176G T177S F180Y
L81V S173P G174K S175P N176G T177S F180Y
A102T S173P G174K S175P N176G T177S F180Y
S173P G174K S175P N176G T177S F180Y T241P
I137E S173P G174K S175P N176G T177S F180Y
S144Q S173P G174K S175P N176G T177S F180Y
Q70N S173P G174K S175P N176G T177S F180Y
D155N G159S S173P G174K S175P N176G T177S F180Y
S173P G174K S175P N176G T177S F180Y S274I
S173P G174K S175P N176G T177S F180Y V286Q
T40D S173P G174K S175P N176G T177S F180Y
S171N S173P G174K S175P N176G T177S F180Y
I121V I137E S173Y G174S S175A F180Y
L81V I137E S173Y G174S S175A F180Y
I137E S173Y G174S S175A F180Y T241P
Q70N I137E S173Y G174S S175A F180Y
I137E S173Y G174S S175A F180Y S274I
I137E S173Y G174S S175A F180Y T297P
I121V I137E S173P G174T S175V T177S F180Y
L81V I137E S173P G174T S175V T177S F180Y
I137E S173P G174T S175V T177S F180Y T241P
Q70N I137E S173P G174T S175V T177S F180Y
I137E S173P G174T S175V T177S F180Y V286Q
I137E S173P G174T S175V T177S F180Y T297P
I137E S171N S173P G174T S175V T177S F180Y
I137E S173P G174T S175A T177S F180Y
I121V I137E S173P G174K S175P N176G T177S F180Y
Q70N I137E S173P G174K S175P N176G T177S F180Y
I137E S173P G174K S175P N176G T177S F180Y S274I
I137E S173P G174K S175P N176G T177S F180Y V286Q
I137E S173P G174K S175P N176G T177S F180Y T297P
I137E S171N S173P G174K S175P N176G T177S F180Y
I137E S173P G174K S175A N176G T177S F180Y
V162R S173Y G174S S175A F180Y
A102T S173Y G174S S175A F180Y
G132I S173P G174T S175V T177S F180Y
S173P G174T S175V T177S F180Y T297P
S173P G174T S175A T177S F180Y
S173P S175A
I137E S144Q S173Y G174S S175A F180Y
T40L I137E S173Y G174S S175A F180Y
I137E S173Y S175A F180Y
I137E S173P S175V T177S F180Y
I137E S173P S175P N176G T177S F180Y
S171N S173Y G174S S175A F180Y
S173P G174K S175A N176G T177S F180Y
I137E S173Y G174S S175A F180Y V286Q
I137E S173P G174T S175V T177S F180Y S274I
T74M I137E S173P
T79I I137E S173P
L81G S173P
L34I I137E S173P
Y39D I137E S173P
T40P I137E S173P
I137E S173P I247M
I137E S173P H256F
I137E S144Q S173P G174T S175V T177S F180Y
I137E S144Q S173P G174K S175P N176G T177S F180Y
S173Y G174S S175P F180Y
I137E S173Y G174S S175P F180Y
S173P G174T S175P T177S F180Y
I137E S173P G174T S175P T177S F180Y
I137E S173P G174T S175P T177S F180Y T297P
V162R S171N S173P G174K S175P N176G T177S F180Y
I137E S171N S173P G174K S175P N176G T177S F180Y T241P
I137E S171N S173P G174K S175P N176G T177S F180Y T297P
I137E S173P S175P F180Y
V162R S173P S175P F180Y
S173P S175P F180Y S274I
S173P S175P F180Y T241P
S173P S175P F180Y T297P
S173P S175P F180Y V286Q
I137E S173Y G174S S175A F180Y
S144Q S173Y G174S S175A F180Y
Q70N S173Y G174S S175A F180Y
I121V S173Y G174S S175A F180Y
L81V S173Y G174S S175A F180Y
G132I S173P G174K S175P N176G T177S F180Y
S173P G174K S175P N176G T177S F180Y T297P

Similarly, it is contemplated that each alpha-amylase variant herein described may further have an improved performance, and/or improved stability, such as an improved wash performance in laundry or in automated dish washing, and/or improved storage stability, compared to any of the listed parent alpha-amylases listed as SEQ ID NO: 2, 3, 4, 5, 6, 7, 8, or 9. Thus, the alpha-amylase variants may further comprise one or more additional substitutions at one or more (e.g., several) other positions. Accordingly, in one embodiment, the alpha-amylase variant of the detergent composition of the present invention further comprises a modification at positions corresponding to positions;

| | |
|---|---|
| 105L, I, F + 206Y, | 195F + 206Y + 208Y + 213T + 214T + 217M, V |
| 105L, I + 206Y + 217I | 195F + 206Y + 208F, L + 213T + 214T + 217V |
| 105F + 206Y + 208Y + 217V + 246V | 195F + 206Y + 213S + 214T |
| 105L + 206F | 195F + 206Y + 208Y + 213S + 214T + 217M |
| 105I + 206Y + 208Y + 217I + 246V | 195F + 206Y + 208F + 213T + 214T + 217M |
| 195F + 213S + 214T | 195F + 206Y + 208Y + 213T + 214T + 217Q |
| 195F + 206Y + 213G + 214T | 195F + 206Y + 213S |
| 195F + 206Y + 208Y + 213T + 214T + 217M | 195F + 213S |
| 195F + 206Y + 208L + 213T + 214T + 217M | 195F + 213G + 214T |
| 206Y, F + 208Y + 217Q | 206Y + 208Y + 217I |
| 206F + 208Y + 217M | 206Y + 208Y |
| 206Y + 217M | 206Y + 208Y + 213A + 217M |
| 206Y + 208Y + 217V + 246V | 206Y + 213G |
| 206Y + 208F + 217V | 206N + 208Y + 217M |
| 206F + 208Y + 217V | 206Y + 246V |
| 206Y + 217I, V | |
| 206F + 208F + 217I | 206Y + 208L + 213S |
| 206F + 217I | 206Y + 217I + 246I |
| 206L + 217V | 206Y + 208F + 217H |
| 206L + 208F + 217I | 195F + 206Y + 208Y |
| 195F + 206Y + 208Y + 213S + 214T | 195F + 206Y + 217V |
| 206Y + 208Y + 213T + 214T + 217V | 195F + 208Y + 213T + 214T + 217V |
| 195F + 206H | 186E + 195F + 202T + 206Y + 210S |
| 195F + 213P | 186E + 195F + 206Y + 210S |
| 195F + 206Y + 208Y + 213T + 214T + 217I | 195F + 206Y + 213P + 214T |
| 63I + 195F + 206Y + 210S | 186E + 195F + 202T + 206Y + 209S |
| 195F + 206Y + 208Y + 213T + 217V | 186E + 195F + 206Y |
| 195F + 206Y + 208Y + 214T + 217V | 63V + 206Y + 241V + 246L |
| 63V + 105F + 206Y | 63V + 206L + 217V |
| 63V + 206F | 63V + 206Y + 246V |
| 63V + 105F + 206Y + 208F + 217I | 63V + 206Y + 217I |
| 63V + 105L + 206Y | 63V + 206Y |
| 63I + 206Y + 241V | 63I + 206Y |
| 208Y + 213A + 217Q | 208Y + 213S + 217M |
| 206F + 246V | 206L + 217V + 246L |
| 195F + 213I + 214P | 213P, S + 214T |
| 213N + 214Q | 213N + 214I |
| 213I + 214P | 213G + 214T |
| 48V + 60V | 213S + 214R |
| 213P + 214L | 213A + 214Q |
| 193A, D, N, S + 195F | 172K + 173Y + 174E |
| 173Y + 174S | 173F + 174Q |
| 179L + 182S + 186Q + 190P | 179L + 182P + 186S, V + 190P |
| 179L + 182C + 186K + 190P | 179L + 190P |
| 179L + 186K, R, S + 190P | 179L + 186H + 190P |
| 182V + 186K | 182S + 186E |
| 182P + 186E | 206Y + 213S |
| 195F + 206Y | 195F + 206Y + 208Y + 213T + 214T |

wherein the numbering is according to SEQ ID NO: 7.

Essential amino acids in a polypeptide may be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. For TY-145 (SEQ ID NO: 1) the catalytic triad comprising the amino acids D35, H72 and S251 is essential for protease activity of the enzyme.

In an embodiment, the detergent composition of the present invention comprises a protease variant as described herein and an alpha-amylase variant as described herein, have an improved stability compared to to detergent composition comprising a parent protease and a parent alpha-amylase having the identical amino acid sequence of the protease variant but not having a substitution at one or more of said specified positions or compared to a protease with SEQ ID NO: 1, 2, 3, 4, 5, 6, 7, 8, or 9, wherein the stability is measured as described in the Examples herein.

In the context of the present invention, any variant, i.e. a protease variant, an alpha-amylase variant, and a lipase variant, have been prepared from a parent enzyme. Such a parent enzyme is defined as a polypeptide comprising or consisting of the amino acid sequences listed as SEQ ID NO: 1, 2, 3, 4, 5, 6, 7, 8, 9, and 10. Thus, the variants have been prepared from a parent enzyme. A parent enzyme may be identified by sequence homology. The homology between two amino acid sequences is in this context described by the parameter “identity” for purposes of the present invention, the degree of identity between two amino acid sequences is determined using the Needleman-Wunsch algorithm as described above. The output from the routine is besides the amino acid alignment the calculation of the “Percent Identity” between the two sequences.

Based on this description it is routine for a person skilled in the art to identify suitable homologous proteases, alpha-amylases, and lipases, which may be modified as described herein.

Substantially homologous parent variants may have one or more (several) amino acid substitutions, deletions and/or insertions, in the present context the term “one or more” is used interchangeably with the term “several”. These changes are preferably of a minor nature, that is conservative amino acid substitutions as described above and other substitutions that do not significantly affect the three-dimensional folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, or protein A (Nilsson et al., 1985, EMBO J. 4: 1075; Nilsson et al., 1991, Methods Enzymol. 198: 3. See, also, in general, Ford et al., 1991, Protein Expression and Purification 2: 95-107.

Although the changes described above preferably are of a minor nature, such changes may also be of a substantive nature such as fusion of larger polypeptides of up to 300 amino acids or more both as amino- or carboxyl-terminal extensions.

The parent enzyme may be (a) a polypeptide having at least 70% sequence identity to the mature polypeptide of SEQ ID NO: 1, 2, 3, 4, 5, 6, 7, 8, 9, or 10.

Accordingly, the parent protease has a sequence identity to the polypeptide with SEQ ID NO: 1 of at least 70%, such as at least 71%, at least 72%, at least 73%, at least 74%, at least 75%, at least 76% at least 77% at least 78% at least 79% at least 80%, at least 81% at least 82% at least 83% at least 84% at least 85%, at least 86% at least 87% at least 88% at least 89%, at least 90%, at least 91%, at least 92%, at least 93%, at least 94%at least 95% identity, at least 96%, at least 97%, at least 98%, or at least 99%, e. g. at least 99.1%, at least 99.2%, at least 99.3%, at least 99.4%, at least 99.5%, at least 99.6, or 100%, which have protease activity.

Accordingly, the parent alpha-amylase has a sequence identity to the polypeptide with SEQ ID NO: 2, 3, 4, 5, 6, 7, 8, or 9 of at least 70%, such as at least 71%, at least 72%, at least 73%, at least 74%, at least 75%, at least 76% at least 77% at least 78% at least 79% at least 80%, at least 81% at least 82% at least 83% at least 84% at least 85%, at least 86% at least 87% at least 88% at least 89%, at least 90%, at least 91%, at least 92%, at least 93%, at least 94%at least 95% identity, at least 96%, at least 97%, at least 98%, or at least 99%, e. g. at least 99.1%, at least 99.2%, at least 99.3%, at least 99.4%, at least 99.5%, at least 99.6, or 100%, which have alpha-amylase activity.

Accordingly, the parent lipase has a sequence identity to the polypeptide with SEQ ID NO: 10 of at least 70%, such as at least 71%, at least 72%, at least 73%, at least 74%, at least 75%, at least 76% at least 77% at least 78% at least 79% at least 80%, at least 81% at least 82% at least 83% at least 84% at least 85%, at least 86% at least 87% at least 88% at least 89%, at least 90%, at least 91%, at least 92%, at least 93%, at least 94%at least 95% identity, at least 96%, at least 97%, at least 98%, or at least 99%, e. g. at least 99.1%, at least 99.2%, at least 99.3%, at least 99.4%, at least 99.5%, at least 99.6, or 100%, which have lipase activity.

The parent enzymes may be a hybrid polypeptide in which a region of one polypeptide is fused at the N-terminus or the C-terminus of a region of another polypeptide and thereby providing the fusion parent enzyme.

The parent enzyme may further be a fusion polypeptide or cleavable fusion polypeptide in which another polypeptide is fused at the N-terminus or the C-terminus of a polypeptide described herein. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent enzyme may be obtained from organisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

Variants present in the detergent composition according to the invention may be prepared by a method for obtaining a variant having the specific enzymatic activity, wherein the method comprises the steps of: (a) introducing into a parent enzyme a modification at one or more (e.g., several) positions as specified herein; and (b) recovering the variant.

The skilled person would know how to prepare a variant. However, variants may be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

In one certain aspect, the present invention provides a detergent composition, wherein the protease and alpha-amylase variants have improved stability in the detergent composition compared to the parent enzyme or compared to an enzyme having the identical amino acid sequence of said variant but not having the substitutions at one or more of said specified positions or compared to a parent enzyme of SEQ ID NO: 1, 2, 3, 4, 5, 6, 7, 8, 9, or 10, wherein stability is measured as described in the Examples herein.

Besides enzymes the detergent compositions according to the invention may comprise additional components. The choice of additional components is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The protease and alpha-amylase variants may be added to a detergent composition in an amount corresponding to 0.001-100 mg of protein, such as 0.01-100 mg of protein, preferably 0.005-50 mg of protein, more preferably 0.01-25 mg of protein, even more preferably 0.05-10 mg of protein, most preferably 0.05-5 mg of protein, and even most preferably 0.01-1 mg of protein per liter of wash liquid.

The protease and alpha-amylase variants may be stabilized using stabilizing agents, which may be selected from the group containing propylene glycol, glycerol, a sugar, a sugar alcohol, lactic acid, boric acid, borate and phenyl boronic acid derivates, such as those where the residue R in the phenyl boronic acid derivative is a C₁-C₆ alkyl group and among these, more preferably, CH₃, CH₃CH₂ or CH₃CH₂CH₂. The residue R in the phenyl boronic acid derivative may also be hydrogen. One example of a phenyl boronic acid derivative is 4-formylphenylboronic acid (4-FPBA) with the following formula:

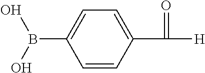 [SMILES: O=Cc1ccc(B(O)O)cc1]

Phenyl boronic acid derivatives may furthermore have other chemical modifications on the phenyl ring, and in particular they can contain one or more methyl, amino, nitro, chloro, fluoro, bromo, hydroxyl, formyl, ethyl, acetyl, t-butyl, anisyl, benzyl, trifluoroacetyl, N-hydroxysuccinimide, t-butyloxycarbonyl, benzoyl, 4-methylbenzyl, thioanizyl, thiocresyl, benzyloxymethyl, 4-nitrophenyl, benzyloxycarbonyl, 2-nitrobenzoyl, 2-nitrophenylsulfenyl, 4-toluenesulfonyl, pentafluorophenyl, diphenylmethyl, 2- chlorobenzyloxycarbonyl, 2,4,5-trichlorophenyl, 2-bromobenzyloxycarbonyl, 9-fluorenylmethyloxycarbonyl, triphenylmethyl, 2,2,5,7,8-pentamethylchroman-6-sulfonyl residues or groups or combinations thereof. All stabilizing agents may be present in the detergent composition of the present invention in all protonated or deprotonated forms. Furthermore, all such compounds, in particular their deprotonated forms, can be associated with cations. Preferred cations in this respect are monovalent or polyvalent, in particular divalent, cations, in particular Na ions (Na⁺), K ions (K⁺), Li ions (Li⁺), Ca ions (Ca²⁺), Mg ions (Mg²⁺), Mn ions (Mn²⁺) and Zn ions (Zn²⁺). The detergent compositions of the present invention may comprise two or more stabilizing agents e.g. such as those selected from the group consisting of propylene glycol, glycerol, 4-formylphenyl boronic acid and borate. One example is a detergent composition of the present invention comprising 4-formylphenyl boronic acid and/or borate. The phenyl boronic acid derivative may be contained in the detergent composition in a quantity of from 0.00001 to 5.0 wt %, preferably from 0.0001 to 3.0 wt %, from 0.001 to 2.0 wt %, from 0.005 to 1.0 wt %, from 0.01 to 0.5 wt %, from 0.02 to 0.3 wt % Preferably, the boric acid/borate is contained in a quantity of from 0.001 to 5.5 wt. % and increasingly preferably of from 0.01 to 4.5 wt. %, from 0.05 to 3.5 and from 0.1 to 3, 0.4 bis 2.49, 0.5 bis 1.5 wt. % in the detergent composition. Addition of a combination of borate and 4-formylphenyl boronic acid has been found to be particularly effective, leading to a high increase in enzyme stability in detergent compositions. Preferably, the boric acid / borate is contained in a quantity of from 0.001 to 5.5 wt. % and increasingly preferably from 0.075 to 4.5 wt. %, from 0.09 to 3.5 and from 0.1 to 2.49 wt. %, and the phenyl boronic acid derivative is contained in a quantity of from 0.001 to 0.08 wt. % and increasingly preferably from 0.003 to 0.06 wt. %, from 0.005 to 0.05 wt. %, from 0.007 to 0.03 wt. % and from 0.009 to 0.01 wt. % in a detergent composition. Particularly preferred is the addition of 4-formylphenyl boronic acid in an amount of 1.0 to 2.0 wt % in combination with 1.0 wt % borate.

The detergent composition according to the invention may comprise protease and alpha-amylase variants which may also be stabilized using peptide aldehydes or ketones such as described in WO 2005/105826 and WO 2009/118375. Another example of detergent compositions according to the invention relates to a detergent composition comprising a protease and alpha-amylase variant as described herein, wherein the detergent formulation is as disclosed in WO 97/07202, which is hereby incorporated by reference.

Other components of the detergent composition according to the present invention may be surfactants. Thus, the detergent composition according to the present invention may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent composition will usually contain from about 1% to about 40% by weight of a cationic surfactant. Non-limiting examples of cationic surfactants include alklydimethylehanolamine quat (ADMEAQ), cetyltrimethylammonium bromide (CTAB), dimethyldistearylammonium chloride (DSDMAC), and alkylbenzyldimethylammonium, and combinations thereof, Alkyl quaternary ammonium compounds, Alkoxylated quaternary ammonium (AQA),

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamide (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent composition will usually contain from about 1% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent composition will usually contain from about 1% to about 40% by weight of a zwitterionic surfactant. Non-limiting examples of zwitterionic surfactants include betaine, alkyldimethylbetaine, and sulfobetaine, and combinations thereof.

Yet another component of the detergent composition according to the present invention is hydrotropes.

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

Thus, the detergent composition according to the present invention may comprise 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonates (STS), sodium xylene sulfonates (SXS), sodium cumene sulfonates (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Another component of a detergent composition may be builders and/or co-builders. The term “builder” may be classified by the test described by M.K. Nagaraja et al., JAOCS, Vol. 61, no. 9 (September 1984), pp. 1475-1478 to determine the minimum builder level required to lower the water hardness at pH 8 from 2.0 mM (as CaCO₃) to 0.10 mM in a solution. The builder may particularly be a chelating agent that forms water-soluble complexes with e.g. calcium and magnesium ions. The term “chelating agents” or “chelators” as used herein, refers to chemicals that form molecules with certain metal ions, inactivating the ions so that they cannot react with other elements thus a binding agent that suppresses chemical activity by forming chelates. Chelation is the formation or presence of two or more separate bindings between a ligand and a single central atom. The ligang may be any organic compound, a silicate or a phosphate. Thus, in one embodiment, the detergent composition according to the present invention may comprise about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with

Ca and Mg. Any builder and/or co-builder known in the art for use in laundry, ADW and hard surfaces cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), iminodiethanol (DEA) and 2,2′,2″-nitrilotriethanol (TEA), and carboxymethylinulin (CMI), and combinations thereof.

The detergent composition according to the present invention may also comprise 0-65% by weight, such as about 5% to about 40%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), etheylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid) (HEDP), ethylenediaminetetrakis(methylene)tetrakis(phosphonic acid) (EDTMPA), diethylenetriaminepentakis(methylene)pentakis(phosphonic acid) (DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP) , iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA) , serine-N,N-diacetic acid (SEDA), isoserine-N,N-diacetic acid (ISDA), phenylalanine-N,N-diacetic acid (PHDA) , anthranilic acid- N,N-diacetic acid (ANDA), sulfanilic acid-N,N-diacetic acid (SLDA) , taurine-N,N-diacetic acid (TUDA) and sulfomethyl-N,N-diacetic acid (SMDA), N-(hydroxyethyl)-ethylidenediaminetriacetate (HEDTA), diethanolglycine (DEG), Diethylenetriamine Penta (Methylene Phosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

Yet another component of the detergent composition may be bleaching systems. Thus, in one embodiment, the detergent composition according to the present invention may comprise 0-10% by weight, such as about 1% to about 5%, of a bleaching system. Any bleaching system known in the art for use in laundry, ADW and hard surfaces cleaning detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. By bleach activator is meant herin a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetyl athylene diamine (TAED), sodium 3,5,5 trimethyl hexanoyloxybenzene sulphonat, diperoxy dodecanoic acid, 4-(dodecanoyloxy)benzenesulfonate (LOBS), 4-(decanoyloxy)benzenesulfonate, 4-(decanoyloxy)benzoate (DOBS), 4-(3,5,5-trimethylhexanoyloxy)benzenesulfonate (ISONOBS), tetraacetylethylenediamine (TAED) and 4-(nonanoyloxy)benzenesulfonate (NOBS), and/or those disclosed in W098/17767. A particular family of bleach activators of interest was disclosed in EP624154 and particulary preferred in that family is acetyl triethyl citrate (ATC). ATC or a short chain triglyceride like Triacin has the advantage that it is environmental friendly as it eventually degrades into citric acid and alcohol. Furthermore acethyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthaloylamino)percapronic acid (PAP). The bleaching system may also include a bleach catalyst. In some embodiments the bleach component may be an organic catalyst selected from the group consisting of organic catalysts having the following formulae:

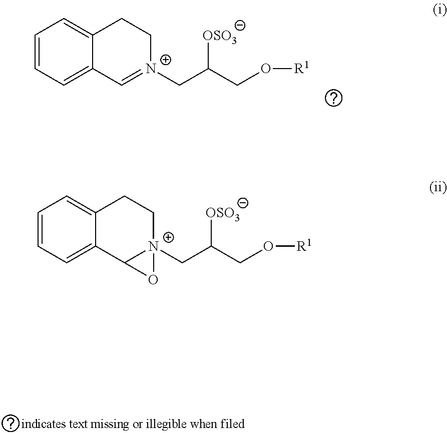

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n- dodecyl, n-tetradecyl, n-hexadecyl, n-octadecyl, iso-nonyl, iso-decyl, iso- tridecyl and iso-pentadecyl. Other exemplary bleaching systems are described, e.g., in WO2007/087258, WO2007/087244, WO2007/087259, WO2007/087242. Suitable photobleaches may for example be sulfonated zinc phthalocyanine

Another component of a detergent composition is polymers. Thus, in one embodiment, the detergent composition according to the invention comprises a polymer.

Accordingly, the detergent composition according to the present invention may comprise 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers , hydrophobically modified CMC (HM-CMC) and silicones, copolymers of terephthalic acid and oligomeric glycols, copolymers of polyethylene terephthalate and polyoxyethene terephthalate (PET-POET), PVP, poly(vinylimidazole) (PVI), poly(vinylpyridin-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

Yet another component of detergent compositions may be fabric hueing agents. Thus, in one embodiment, the detergent composition according to the invention comprises a fabric hueing agent.

The detergent composition according to the present invention may also comprise fabric hueing agents such as dyes or pigments which when formulated in detergent compositions can deposit onto a fabric when said fabric is contacted with a wash liquor comprising said detergent compositions thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). A detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257, WO2007/087243.

### Further Enzymes

In one embodiment, the detergent composition according to the invention comprises one or more enzymes, such as at least two enzymes, more preferred at least three, four or five enzymes.

Preferably, the enzymes have different substrate specificity, e.g., proteolytic activity, amylolytic activity, lipolytic activity, hemicellulytic activity or pectolytic activity.

The detergent composition according to the invention may comprise one or more additional enzymes such as carbohydrate-active enzymes like carbohydrase, pectinase, mannanase, amylase, cellulase, arabinase, galactanase, xylanase, or protease, lipase, a, cutinase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes NS), Celluclean™ (Novozymes NS), Celluclean Classic™ (Novozymes NS), Cellusoft™ (Novozymes A/S), Whitezyme™ (Novozymes NS), Clazinase™, and Puradax HA™ (Genencor International Inc.), and KAC-500(B)™ (Kao Corporation).

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes NS).

Suitable additional proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN', subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens. Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN' numbering. More preferred the protease variants may comprise the mutations: S3T, V4I, S9R, A15T, K27R, *36D, V68A, N76D, N87S,R, *97E, A98S, S99G,D,A, S99AD, S101G,M,R S103A, V104I, Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes NS), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Eraser®, Opticlean® and Optimase® (Danisco/DuPont), Axapem™ (Gist-Brocases N.V.), BLAP (sequence shown in FIG. 29 of U.S. Pat. No. 5352604) and variants hereof (Henkel AG) and KAP (Bacillus alkalophilus subtilisin) from Kao.

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the 554V variant used in the commercial product Gentle Power Bleach from Huntsman Textile Effects Pte Ltd (WO10/100028).

Suitable additional amylases which can be used together with the variants of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181.

Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions:

N21D, D97N, V1281 K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes NS), and Rapidase™, Purastar™/Effectenz™, Powerase, Preferenz S1000, Preferenz S100 and Preferenz S110 (from Genencor International Inc./DuPont).

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes NS).

A detergent composition according to the invention may also comprise additional enzymes such as pectate lyases e.g. Pectawash™, chlorophyllases etc.

The detergent enzyme(s) may be included in the detergent composition according to the invention by adding separate additives containing one or more enzymes, or by adding a combined additive comprising all of these enzymes. A detergent additive, i.e., a separate additive or a combined additive, may be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. No. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized.

Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

The detergent composition according to the invention may also comprise dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc. The detergent composition according to the invention may also comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition. A detergent composition according to the invention may preferably also comprise additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1.2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %. The detergent composition according to the invention may also comprise one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof. The detergent composition according to the invention may also comprise one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

Thus, in one particular embodiment, the detergent composition further comprises at least one chelating agent; at least one surfactant; at least one sulfonated polymer; at least one hydrotrope; at least one builder and/or co-builder; at least one perfume; and/or at least one kind of bleaching system.

### Formulation of Detergent Products

The detergent composition according to the invention may be in any convenient form, e.g., a bar, a homogenous tablet, a tablet having two or more layers, a regular or compact powder, a granule, a paste, a gel, or a regular, compact or concentrated liquid.

Thus, in one embodiment, the detergent composition according to the present invention is a liquid laundry detergent composition, a powder laundry detergent composition, a liquid dishwash detergent composition, or a powder dishwash detergent composition.

Detergent formulation forms: Layers (same or different phases), Pouches, versus forms for Machine dosing unit.

Pouches may be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxyprpyl methyl cellulose (HPMC). Preferably the level of polymer in the film for example PVA is at least about 60%. Preferred average molecular weight will typically be about 20,000 to about 150,000. Films can also be of blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticisers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids. Ref: (US2009/0011970 A1)

Detergent ingredients may be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent , which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Methods and Uses

A detergent composition according to the invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. Thus, in one embodiment, the detergent composition is a liquid laundry detergent composition, a powder laundry detergent composition, a liquid dishwash detergent composition; or a powder dishwash detergent composition.

A cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. A process for laundering of fabrics and/or garments may be a process comprises treating fabrics with a washing solution containing a detergent composition, and at least one protease variant. A cleaning process or a textile care process can for example be carried out in a machine washing process or in a manual washing process. The washing solution can for example be an aqueous washing solution containing a detergent composition.

The fabrics and/or garments subjected to a washing, cleaning or textile care process may be conventional washable laundry, for example household laundry. Preferably, the major part of the laundry is garments and fabrics, including knits, woven, denims, non-woven, felts, yarns, and towelling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

Typical detergent compositions include various components in addition to the enzymes, these components have different effects, some components like the surfactants lower the surface tension in the detergent, which allows the stain being cleaned to be lifted and dispersed and then washed away, other components like bleach systems remove discolor often by oxidation and many bleaches also have strong bactericidal properties, and are used for disinfecting and sterilizing. Yet other components like builder and chelator softens, e.g., the wash water by removing the metal ions form the liquid.

The enzyme compositions may further comprise at least one or more of the following: a surfactant, a builder, a chelator or chelating agent, bleach system or bleach component in laundry or dish wash.

The amount of a surfactant, a builder, a chelator or chelating agent, bleach system and/or bleach component may be reduced compared to amount of surfactant, builder, chelator or chelating agent, bleach system and/or bleach component used without the added protease variant of the invention. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of protease variants of the invention, such as a conventional amount of such component. Detergent compositions may also be composition which is free of at least one component which is a surfactant, a builder, a chelator or chelating agent, bleach system or bleach component and/or polymer.

### Washing Method

Detergent composition according to the invention is ideally suited for use in laundry applications. Thus, in one aspect, the present invention relates to a method of laundering, comprising laundering a garment with a detergent composition as described herein, preferably at a temperature of 40° C. or less, or more preferably at a temperature of 30° C. or less, or even more preferably at a temperature of 20° C. or less.

These methods include a method for laundering a fabric. The method comprises the steps of contacting a fabric to be laundered with a cleaning laundry solution comprising a detergent composition. The fabric may comprise any fabric capable of being laundered in normal consumer use conditions. The solution preferably has a pH from about 5.5 to about 11.5. The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH from about 5.0 to about 11.5, or from about 6 to about 10.5, about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, about 8 to about 11, about 8 to about 10, about 8 to about 9, about 9 to about 11, about 9 to about 10, about 10 to about 11, preferably about 5.5 to about 11,5.

In particular embodiments, the washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 16° dH, under typical US wash conditions about 6° dH, and under typical Asian wash conditions, about 3° dH.

The compositions for use in the methods described above may further comprises at least one additional enzyme as set forth in the “other enzymes” section above, such as an enzyme selected from the group of hydrolases such as proteases, lipases and cutinases, carbohydrases such as amylases, cellulases, hemicellulases, xylanases, and pectinase or a combination hereof.

The present invention is further described by the following examples that should not be construed as limiting the scope of the invention.

### EXAMPLES

### Materials and Methods

### Automatic Mechanical Stress Assay (AMSA) for Laundry

In order to assess the wash performance in laundry washing experiments are performed, using the Automatic Mechanical Stress Assay (AMSA). With the AMSA, the wash performance of a large quantity of small volume enzyme-detergent solutions can be examined. The AMSA plate has a number of slots for test solutions and a lid firmly squeezing the laundry sample, the textile to be washed against all the slot openings. During the washing time, the plate, test solutions, textile and lid are vigorously shaken to bring the test solution in contact with the textile and apply mechanical stress in a regular, periodic oscillating manner. For further description see WO02/42740 especially the paragraph “Special method embodiments” at page 23-24.

The wash performance is measured as the brightness of the colour of the textile washed. Brightness can also be expressed as the intensity of the light reflected from the sample when illuminated with white light. When the sample is stained the intensity of the reflected light is lower, than that of a clean sample. Therefore the intensity of the reflected light can be used to measure wash performance.

Colour measurements are made with a professional flatbed scanner (Kodak iQsmart, Kodak, Midtager 29, DK-2605 Brondby, Denmark), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r+g²+b²}

The experiment was conducted under the experimental conditions as specified in table 1 below.

TABLE 1
AMSA Experimental Conditions

| | |
|---|---|
| Detergent | Model detergent B |
| Detergent dosage | 3.33 g/L |
| Test solution volume | 160 μL |
| Wash time | 20 minutes |
| Temperature | 20° C. |
| Water hardness | 15° dH |
| Protease concentration | 30 nM |
| in test solution | |
| Amylase concentration | 0.2 ppm |
| in test solution | |
| Test material | Chocolate milk and soot swatch (PC-03) |
| | Rice Starch, coloured (CS-28) |

Water hardness was adjusted to 15° dH by addition of CaCl₂, MgCl₂, and NaHCO₃ (Ca²⁺:Mg²⁺:CO₃²⁻=4:1:7.5) to the test system. The swatch textiles were flushed in tap water and dried after washing. The performance of an enzyme variant is measured as the brightness of the colour of the textile washed with that specific protease. Brightness can also be expressed as the intensity of the light reflected from the sample when illuminated with white light. When the sample is stained the intensity of the reflected light is lower, than that of a clean sample. Therefore the intensity of the reflected light can be used to measure wash performance of a protease. Color measurements were made with a professional flatbed scanner (EPSON EXPRESSION 10000XL, Atea NS, Lautrupvang 6, 2750 Ballerup, Denmark), which is used to capture an image of the washed textile swatches.

TABLE 2
Composition of model detergents and test materials
Model detergent and test materials were as follows:

| | Ingredient | wt % |
|---|---|---|
| Laundry | (C10-C13)alkylbenzene sulfonic acid | 7.2 |
| liquid | sodium lauryl ether sulfate | 10.6 |
| model B | cocoa fatty acid | 2.75 |
| detergent | soy fatty acid | 2.75 |
| | alcohol ethoxylate with 8 mol EO | 6.6 |
| | sodium hydroxide | 1.1 |
| | ethanol | 3 |
| | propane-1,2-diol | 6 |
| | glycerol | 1.7 |
| | triethanolamine | 3.3 |
| | sodium formiate | 1 |
| | sodium citrate | 2 |
| | diethylenetriaminepentakis(methylene)pen- | 0.5 |
| | takis(phosphonic acid), heptasodium salt | |
| | copoly(acrylic acid/maleic acid), sodium salt | 0.5 |
| | deionized water | 51 |

### General Molecular Biology Methods:

Unless otherwise mentioned the DNA manipulations and transformations were performed using standard methods of molecular biology (Sambrook et al. (1989); Ausubel et al. (1995); Harwood and Cutting (1990).

### Protease Activity Assays:

### 1) Suc-AAPF-pNA Activity Assay:

The proteolytic activity can be determined by a method employing the Suc-AAPF-PNA substrate. Suc-AAPF-PNA is an abbreviation for N-Succinyl-Alanine-Alanine-Proline-Phenylalanine-p-Nitroanilide, and it is a blocked peptide which can be cleaved by endo-proteases. Following cleavage a free PNA molecule is liberated and it has a yellow colour and thus can be measured by visible spectrophotometry at wavelength 405nm. The Suc-AAPF-PNA substrate is manufactured by Bachem (cat. no. L1400, dissolved in DMSO).

The protease sample to be analyzed was diluted in residual activity buffer (100 mM Tris pH8.6). The assay was performed by transferring 60p1 of diluted enzyme samples to 96 well microtiter plate and adding 140 μl substrate working solution (0.72 mg/ml in 100 mM Tris pH8.6). The solution was mixed at room temperature and absorption is measured every 20 sec. over 5 minutes at OD 405 nm.

The slope (absorbance per minute) of the time dependent absorption-curve is directly proportional to the specific activity (activity per mg enzyme) of the protease in question under the given set of conditions. The protease sample should be diluted to a level where the slope is linear.

Alpha-Amylase Activity Assay—pNP-G7 Assay

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No.11876473).

### Reagents:

The G7-pNP substrate from this kit contains 22 mM 4,6-ethylidene- G7-pNP and 52.4 mM HEPES (2-[4-(2-hydroxyethyl)-1-piperazinyl]-ethanesulfonic acid), pH 7.0) . The alpha-Glucosidase reagent contains 52.4 mM HEPES, 87 mM NaCl, 12.6 mM MgCl₂, 0.075 mM CaCl₂, ≧4 kU/L alpha-glucosidase).

The substrate working solution is made by mixing 1 mL of the alpha-Glucosidase reagent with 0.2 mL of the G7-pNP substrate. This substrate working solution is made immediately before use. Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl2, pH8.0.

### Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Alpha-Amylase Activity Assay—Phadebas Activity Assay

The alpha-amylase activity may also be determined by a method using the Phadebas substrate (from for example Magle Life Sciences, Lund, Sweden). A Phadebas tablet includes interlinked starch polymers that are in the form of globular microspheres that are insoluble in water. A blue dye is covalently bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 620 nm. The concentration of blue is proportional to the alpha-amylase activity in the sample.

The alpha-amylase sample to be analyzed is diluted in activity buffer with the desired pH. Two substrate tablets are suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl M NaOH and mix. Centrifuge MTP for 5 minutes at 4000×g. Transfer 100 μl to new MTP and measure absorbance at 620 nm.

The alpha-amylase sample should be diluted so that the absorbance at 620nm is between 0 and 2.2, and is within the linear range of the activity assay.

### Alpha-Amylase Activity Assay—Amylazyme Activity Assay

The alpha-amylase activity may also be determined by a method using the Amylazyme substrate (Megazyme® Amylazyme Test, supplied by Megazyme for the assay of cereal and bacterial amylases) comprising AZCL-amylose, which has been mixed with lactose and magnesium stearate and tabletted. A blue dye is covalently bound to these microspheres. The interlinked amylose polymers in the microsphere are degraded at a speed that is proportional to the alpha-amylase activity. When the alpha-amylase degrades the starch polymers, the released blue dye is water soluble and concentration of dye may be determined by measuring absorbance at 590 nm. The concentration of blue is proportional to the alpha-amylase activity in the sample.

The alpha-amylase sample to be analyzed is diluted in activity buffer with the desired pH. Two substrate tablets are suspended in 5 mL activity buffer and mixed on magnetic stirrer. During mixing of substrate 150 μl is transferred to a microtiter plate (MTP) or PCR-MTP. Next, 25 μl diluted amylase sample is added to 150 μl substrate and mixed. The mixture is incubated for 10 minutes at 37° C. The reaction is stopped by adding 25 μl 1M NaOH and mixed. MTP is centrifuged for 5 minutes at 4000×g, followed by transferring 100 μl to a new MTP and absorbance is measured at 590 nm.

### Example 1

### Preparation and Testing of Variants Comprised in the Detergent Composition of the Invention

Reparation and Expression of variants

Site-directed variants were constructed of the parent protease (SEQ ID NO: 1) and parent alpha-amylases (SEQ ID NO: 2, 3, 4, 5, 6, and 9) comprising specific insertions/deletions/substitutions in the regions as defined elsewhere herein. The variants were made by traditional cloning of DNA fragments (Sambrook et al., Molecular Cloning: A Laboratory Manual, 2nd Ed., Cold Spring Harbor, 1989) using PCR together with properly designed mutagenic oligonucleotides that introduced the desired mutations in the resulting sequence. Mutagenic oligos were synthesized corresponding to the DNA sequence flanking the desired site(s) of mutation, separated by the DNA base pairs defining the insertions/deletions/substitutions. In this manner, the variants listed in table 2a below were constructed and produced.

### Fermentation of Variants

Fermentation may be performed by methods well known in the art or as follows. A B. subtilis strain harboring the relevant expression plasmid was streaked on a LB-agar plate with a relevant antibiotic (6 μg/ml chloramphenicol), and grown overnight at 37° C.

The colonies were transferred to 100 ml PS-1 media supplemented with the relevant antibiotic in a 500 ml shaking flask containing a rich media (e.g. PS-1: 100 g/L Sucrose (Danisco cat.no. 109-0429), 40 g/L crust soy (soy bean flour), 10 g/L Na₂HPO₄.12H₂O (Merck cat.no. 6579), 0.1 ml/L Pluronic PE 6100 (BASF 102-3098)). Cultivation typically takes 4 days at 30° C. shaking with 220 rpm. Cells and other undissolved material were removed from the fermentation broth by centrifugation at 4500 rpm for 20-25 minutes. Afterwards the supernatant was filtered to obtain a clear solution.

### Example 2

### Wash Testing of Protease Variants Together with Amylases by Automatic Mechanical Stress Assay (AMSA)

Washing experiments are performed in order to assess the wash performance of selected protease variants together with amylases in laundry detergent. A group of 10 proteases together with 4 different amylases. were tested using the Automatic Mechanical Stress Assay (AMSA). The results are shown in tables 3 and 4.

TABLE 3
AMSA wash performance data on chocolate/soot (PC-03) textile for protease variants
with and without amylase relative to their parent protease backbone (PTY00).

| | | | AMSA Wash Performance on PC-03 | | | |
|---|---|---|---|---|---|---|
| | | | Textile | | | |
| | | | (relative to parent protease backbone) | | | |
| ID | Mutations | Amylase | Amylase 1 | Amylase 2 | Amylase 3 | Amylase 4 |
| PTY00 | | − | 1.0 | 1.0 | 1.0 | 1.0 |
| PTY00 | | + | 1.1 | 0.7 | 1.0 | 1.1 |
| PTY01 | S173P | − | 0.7 | 0.9 | 1.1 | 1.1 |
| PTY01 | S173P | + | 1.1 | 0.8 | 1.0 | 0.9 |
| PTY02 | S173P S175A | − | 1.1 | 0.8 | 1.1 | 0.7 |
| PTY02 | S173P S175A | + | 1.0 | 0.7 | 1.5 | 0.8 |
| PTY03 | S173P S175P | − | 0.8 | 0.5 | 1.1 | 0.5 |
| PTY03 | S173P S175P | + | 1.2 | 0.5 | 1.1 | 0.9 |
| PTY04 | S173P S175P F180Y | − | 0.7 | 0.2 | 0.6 | 0.5 |
| PTY04 | S173P S175P F180Y | + | 0.8 | 0.9 | 1.2 | 1.1 |
| PTY05 | S171D S173P S175P | − | 0.8 | 0.9 | 1.1 | 0.9 |
| PTY05 | S171D S173P S175P | + | 1.0 | 1.0 | 0.8 | 1.0 |
| PTY06 | S171K S173P S175P | − | 0.8 | 1.1 | 1.0 | 1.0 |
| PTY06 | S171K S173P S175P | + | 1.0 | 1.0 | 1.0 | 1.0 |
| PTY07 | S171N S173P S175P | − | 0.8 | 0.5 | 0.8 | 0.7 |
| PTY07 | S171N S173P S175P | + | 1.1 | 0.6 | 1.1 | 0.9 |
| PTY09 | S173P S175P G179T | − | 1.1 | 0.9 | 0.9 | 1.1 |
| PTY09 | S173P S175P G179T | + | 1.1 | 1.1 | 0.9 | 1.1 |
| PTY10 | S171N S173P S175P F180Y | − | 0.7 | 0.7 | 0.6 | 0.7 |
| PTY10 | S171N S173P S175P F180Y | + | 0.7 | 0.6 | 0.7 | 0.6 |

TABLE 4
AMSA wash performance data on rice starch (CS-28) textile for protease variants with
and without amylase relative to their parent amylase backbone (1-4).

| | | | AMSA Wash Performance on CS-28 | | | |
|---|---|---|---|---|---|---|
| | | | Textile | | | |
| | | | (relative to parent amylase backbone) | | | |
| ID | Mutations | Amylase | Amylase 1 | Amylase 2 | Amylase 3 | Amylase 4 |
| | | + | 1.0 | 1.0 | 1.0 | 1.0 |
| PTY00 | | − | 4.5 | 0.6 | 0.5 | 0.3 |
| PTY00 | | + | 7.2 | 1.1 | 1.1 | 0.9 |
| PTY01 | S173P | − | 1.1 | 0.2 | 0.2 | 0.1 |
| PTY01 | S173P | + | 0.8 | 0.9 | 0.9 | 0.9 |
| PTY02 | S173P S175A | − | 0.0 | 0.0 | 0.0 | 0.0 |
| PTY02 | S173P S175A | + | 0.0 | 0.9 | 0.8 | 0.9 |
| PTY03 | S173P S175P | − | 0.0 | 0.0 | 0.0 | 0.0 |
| PTY03 | S173P S175P | + | 0.0 | 0.9 | 0.8 | 1.0 |
| PTY04 | S173P S175P F180Y | − | 0.0 | 0.1 | 0.0 | 0.0 |
| PTY04 | S173P S175P F180Y | + | 1.7 | 1.0 | 1.1 | 1.1 |
| PTY05 | S171D S173P S175P | − | 0.0 | 0.1 | 0.0 | 0.0 |
| PTY05 | S171D S173P S175P | + | 1.2 | 0.9 | 0.9 | 1.0 |
| PTY06 | S171K S173P S175P | − | 0.0 | 0.1 | 0.0 | 0.0 |
| PTY06 | S171K S173P S175P | + | 0.3 | 0.9 | 0.9 | 0.9 |
| PTY07 | S171N S173P S175P | − | 0.0 | 0.1 | 0.0 | 0.0 |
| PTY07 | S171N S173P S175P | + | 0.2 | 0.9 | 1.0 | 0.9 |
| PTY09 | S173P S175P G179T | − | 1.0 | 0.0 | 0.1 | 0.0 |
| PTY09 | S173P S175P G179T | + | 1.9 | 1.0 | 1.1 | 1.0 |
| PTY10 | S171N S173P S175P F180Y | − | 0.1 | 0.1 | 0.0 | 0.0 |
| PTY10 | S171N S173P S175P F180Y | + | 1.2 | 0.9 | 0.9 | 1.0 |

1. A detergent composition comprising (i) at least one protease variant comprising
a substitution
at one or more positions corresponding to positions 171, 173, 175, 179, or 180 of SEQ ID NO:
1, wherein said protease variant has a sequence identity of at least 75% but less than 100% to SEQ ID NO: 1, and wherein the protease variant has protease activity, and (ii) at least one alpha-amylase having the amino acid sequence of SEQ ID NO: 2, 3, 4, 5, 6, 7, 8, or 9, or a variant thereof having a sequence identity of at least 75% but less than 100% to SEQ ID NO: 4, 5, 6, 7, 8, or 9, and wherein said alpha-amylase variant has alpha-amylase activity.

2. The detergent composition according to claim 1, wherein said at least one protease variant comprises a substitution in at least one position corresponding to positions 173, 175, or 180 of SEQ ID NO: 1.

3. The detergent composition according to claim 1, wherein said at least one protease variant comprises a substitution in at least one position corresponding to positions 171, 173, 175, 179, or 180, and wherein
a. the amino acid in the position corresponding to position 171 of SEQ ID NO: 1 is selected from the group consisting of W, K, E,D and N; and/or
b. the amino acid in the position corresponding to position 173 of SEQ ID NO: 1 is P; and/or
c. the amino acid in the position corresponding to position 175 of SEQ ID NO: 1 is selected from the group consisting of A, V, and P; and/or
d. the amino acid in the position corresponding to position 179 of SEQ ID NO: 1 is selected from the group consisting of C, V, Q, S, T, E, H, K, M, N, Y, and A; and/or
e. the amino acid in the position corresponding to position 180 of SEQ ID NO: 1 is Y.

4. The detergent composition according to claim 1, wherein said substitution is selected from S173P, S175P or F180Y when referring to SEQ ID NO: 1.

5. The detergent composition according to claim 1, wherein said at least one alpha-amylase is a variant of a parent alpha-amylase of SEQ ID NO: 2, 3, 4, 5, 6, 7, 8, or 9 , and wherein said alpha-amylase variant has alpha-amylase activity.

6. The detergent composition according to claim 4, wherein said at least one alpha-amylase variant is selected from the group consisting of:
a. an alpha-amylase comprising a deletion and/or a substitution at two or more positions corresponding to positions 181, 182, 183, and 184 of SEQ ID NO: 4, and wherein said at least one alpha-amylase variant has at least 75% but less than 100% sequence identity to SEQ ID NO: 2;
b. an alpha-amylase comprising one or more modifications in the following positions: 9, 118, 149, 182, 186, 195, 202, 257, 295, 299, 320, 323, 339, 345, and 458 of SEQ ID NO: 5, and wherein said at least one alpha-amylase variant has at least 75% but less than 100% sequence identity to SEQ ID NO: 3;
c. an alpha-amylase comprising one or more modifications in the following positions: 140, 195, 206, 243, 260, and 476 of SEQ ID NO: 2, and wherein said at least one alpha-amylase variant has at least 75% but less than 100% sequence identity to SEQ ID NO: 2;
d. an alpha-amylase comprising one or more modifications in the following positions: 180, 181, 243, and 475 of SEQ ID NO: 4, and wherein said at least one alpha-amylase variant has at least 75% but less than 100% sequence identity to SEQ ID NO: 4;
e. an alpha-amylase comprising one or more modifications in the following positions: 178, 179, 187, 203, 458, 459, 460, and 476 of SEQ ID NO: 5, and wherein said at least one alpha-amylase variant has at least 75% but less than 100% sequence identity to SEQ ID NO: 5;
f. an alpha-amylase comprising a modification in position 202 of SEQ ID NO: 6, and wherein said at least one alpha-amylase variant has at least 75% but less than 100% sequence identity to SEQ ID NO: 6;
g. an alpha-amylase comprising one or more modifications in the following positions: 405, 421, 422, and 428 of SEQ ID NO: 7, and wherein said at least one alpha-amylase variant has at least 75% but less than 100% sequence identity to SEQ ID NO: 7; and
h. an alpha-amylase comprising one or more modifications in the following positions: 48, 49, 107, 156, 181, 190, 209, and 264 of SEQ ID NO: 9, and wherein said at least one alpha-amylase variant has at least 75% but less than 100% sequence identity to SEQ ID NO: 9.

7. The detergent composition according to claim 5, wherein said at least one alpha-amylase variant is selected from the group consisting of;
a. an alpha-amylase comprising a deletion in the positions corresponding to D183 and G184 of SEQ ID NO: 2;
b. an alpha-amylase comprising the modifications R118K, D183*, G184*, N195F, R320K, and R458K of SEQ ID NO:3;
c. an alpha-amylase comprising the modifications M9L, R118K, G149A, G182T, G186A, D183*, G184*, N195F, M202L, T257I, Y295F, N299Y, R320K, M323T, A339S, E345R, and R458K of SEQ ID NO: 3;
d. an alpha-amylase comprising the modifications D183*, G184*, W140Y, N195F, and I206Y of SEQ ID NO: 2;
e. an alpha-amylase comprising the modifications R180*, S181*, S243Q, G475K, and CBM* of SEQ ID NO: 4;
f. an alpha-amylase comprising the modifications R178*, G179*, E187P, I203Y, R458N,T459S, D460T, and G476K of SEQ ID NO: 5;
g. an alpha-amylase comprising the substitution M202L of SEQ ID NO: 6;
h. an alpha-amylase comprising the modification M197T of SEQ ID NO: 9;
i. an alpha-amylase comprising the modifications H156Y, A181T, N190F, A209V, and Q264S of SEQ ID NO: 9; and
j. an alpha-amylase comprising the modifications G48A, T49I, G107A, H156Y, A181T, N190F, L201F, A209V, and Q264S of SEQ ID NO: 9.

8. The detergent composition according to claim 1, further comprising one or more lipases having the amino acid sequence of SEQ ID NO: 10, or a variant thereof having a sequence identity of at least 75% but less than 100% to SEQ ID NO: 10, and wherein said variant has lipase activity.

9. The detergent composition according to claim 1, wherein the number of modifications in said variants is 1 to 20.

10. The detergent composition according to claim 1, further comprising at least one chelating agent; at least one surfactant; at least one sulfonated polymer; at least one hydrotrope; at least one builder and/or co-builder; at least one perfume; and/or at least one kind of bleaching system.

11. The detergent composition according to claim 1, wherein said detergent composition is a liquid laundry detergent composition, a powder laundry detergent composition, a liquid dishwash detergent composition, or a powder dishwash detergent composition.

12. A method of laundering, comprising laundering a fabric with a detergent composition according to claim 1 at a temperature of 40° C. or less, 30° C. or less, or 20° C. or less.


SEQUENCE LISTING (10 sequences)

SEQ ID NO: 1 - protein, 311 aa, Bacillus sp. [sequence omitted]

SEQ ID NO: 2 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 3 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 4 - protein, 484 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYTFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 5 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 6 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 7 - protein, 483 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 8 - protein, 485 aa, Bacillus sp.
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 9 - protein, 481 aa, Bacillus sp.
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWYWYHFDGTDWDESRKLNRIYKFQGKTWDWEVSNEFGNYDYLMYADIDYDHPDVVAEIKRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 10 - protein, 269 aa, Thermomyces lanuginosus [sequence omitted]